(19) United States
(12) Patent Application Publication
(10) Pub. No.: US 2015/0104428 A1
(43) Pub. Date: Apr. 16, 2015

(54) Compositions and Treatment Methods for Mesenchymal Stem Cell-Induced Immunoregulation

(71) Applicant: University of Southern California, Los Angeles, CA (US)

(72) Inventors: Songtao Shi, Thousand Oaks, CA (US); Kentaro Akiyama, Pasadena, CA (US); Chider Chen, San Gabriel, CA (US)

(73) Assignee: University of Southern California, Los Angeles, CA (US)

(21) Appl. No.: 14/389,322

(22) PCT Filed: Mar. 29, 2013
(86) PCT No.: PCT/US13/34719

§ 371 Date: Sep. 29, 2014

Related U.S. Application Data
(60) Provisional application No. 61/618,636, filed on Mar. 30, 2012.

(51) Int. Cl. A61K 35/28

(52) U.S. Cl. CPC A61K 35/28; A61K 2035/122
USPC 424/93.7; 435/325; 435/372

(57) ABSTRACT

Mesenchymal Stem Cells (MSCs), including bone marrow-derived MSCs (BMMSCs) expressing Fas and FasL, and secreting MCP-1 are disclosed. Also disclosed are methods for upregulating regulatory T cells in a subject by administering MSCs, including BMMSCs. Also disclosed are methods for treating systemic sclerosis or colitis in a subject by administering MSCs, including BMMSCs.

### CROSS-REFERENCE TO RELATED APPLICATIONS

This application claims the benefit of U.S. Provisional patent application No. 61/618,636, entitled Compositions and Treatment Methods for Mesenchymal Stem Cell-Induced Immunoregulation, filed on Mar. 30, 2012, with the first named inventor/applicant name of Songtao Shi, the entire contents of which is hereby incorporated by reference in its entirety.

### STATEMENT REGARDING FEDERALLY SPONSORED RESEARCH OR DEVELOPMENT

This invention was made with government support under Contract Nos. R01DE017449, R10 DE019932, and R10 DE019413 from the National Institute of Dental and Craniofacial Research, National Institutes of Health, Department of Health and Human Services. The government has certain rights in the invention.

### FIELD OF THE INVENTION

The present invention is directed to compositions and treatment methods for Mesenchymal Stem Cell-induced immunoregulation.

### BACKGROUND OF THE INVENTION

Various tissues, including bone marrow, contain stem-like precursors for non-hematopoietic cells, such as osteoblasts, chondrocytes, adipocytes and myoblasts (Owen et al., 1988, in Cell and Molecular Biology of Vertebrate Hard Tissues, Ciba Foundation Symposium 136, Chichester, UK, pp. 42-60; Caplan, 1991, J. Orthop. Res 9:641-650; Prockop, 1997, Science 276:71-74). The non-hematopoetic precursor cells of these various tissues are referred to as Mesenchymal stem cells (MSCs). In vivo MSCs are diverse and subpopulations express a variety of different sets of proteins and surface antigens. MSCs display immunomodulatory properties by inhibiting proliferation and function of several major immune cells, such as dendritic cells, T and 13 lymphocytes, and natural killer (NK) cells (Nauta and Fibbe, 2007; Uccelli et al., 2007, 2008; Aggarwal and Pittenger, 2005). These properties have prompted researchers to investigate mechanisms by which MSCs ameliorate a variety of immune disorders (Nauta and Fibbe, 2007; Bernardo et al., 2009). In fact, MSC-based therapy has been successfully applied in various human diseases, including graft versus host disease (GvHD), systemic lupus erythematosus (SLE), diabetes, rheumatoid arthritis, autoimmune encephalomyelitis, inflammatory bowel disease, and multiple sclerosis (Aggarwal and Pittenger, 2005; Le Blanc et al., 2004; Chen et al., 2006; Polchert et al., 2008; Sun et al., 2009; Lee et al., 2006; Augello et al., 2007; Parekkadan et al., 2008; Zappia et al., 2005; González et al., 2009; Liang et al., 2009). The immunosuppressive properties of MSCs are associated with the production of cytokines, such as interleukin 10 (IL10), nitric oxide (NO), indoleamine 2,3-dioxygenase (TDO), prostaglandin (PG) E2, and TSG-6 (Batten et al., 2006; Zhang et al., 2010; Ren et al., 2008, Sato et al., 2007; Meisel et al., 2004; Aggarwal and Pittenger, 2005; Choi et al., 2011; Roddy et al., 2011; Nemeth et al., 2009). In addition, MSC-induced immune tolerance involves upregulation of CD4⁺CD25⁺Foxp3⁺ regulatory T cells (Tregs) and downregulation of proinflammatory T helper 17 (Th17) cells (Sun et al., 2009; González et al., 2009; Park et al., 2011). However, the detailed mechanism of MSC-based immunotherapy is not fully understood. In this study, we show that MSC-induced T cell apoptosis through Fas signaling is required for MSC-mediated therapeutic effects in SS and experimental colitis in mice.

MSC-based immune therapies have been widely used in preclinical animal models and clinics in an attempt to cure a variety of immune-related diseases (Kikuiri et al., 2010; Schurgers et al. 2010; Park et al., 2011; Liang et al., 2010 and 2011; Wang et al., 2011; Zhou et al., 2008). Many factors contributing to MSC-based immune therapies have been reported (Augello et al., 2005; Aggarwal and Pittenger, 2005; Selmani et al., 2008; Nasef et al., 2008; Ren et al., 2010; Choi et al., 2011; Roddy et al., 2011). However, the detailed mechanism that governs efficacy of MSC-based immune therapies is not fully understood. It was suggested that the inhibitory effect of MSCs on T cell proliferation resulted from the induction of T cell apoptosis, which is associated with the conversion of tryptophan into kynurenine by indoleamine 2,3-dioxygenase (Plumas et al., 2005).

### SUMMARY OF THE INVENTION

Systemic infusion of mesenchymal stem cells (MSCs), preferably bone marrow-derived mesenchymal stem cells (BMMSCs), shows therapeutic effects on a variety of autoimmune diseases, but the underlying mechanisms of MSC-based immunoregulation are not fully understood. Here we showed that systemic infusion of BMMSCs induced a transient T cell apoptosis via the Fas Ligand (FasL)-dependent Fas pathway by which diseased phenotypes in fibrillin-1 mutated systemic sclerosis (SS) and dextran sulfate sodium-induced experimental colitis mice were ameliorated. On the other hand, FasL^{-/-} BMMSCs did not induce T cell apoptosis in recipients, hence, were incapable of ameliorating SS and colitis, whereas overexpression of FasL in FasL^{-/-} BMMSCs rescued these phenotypes. Unexpectedly, Fas^{-/-} BMMSCs with normal FasL expression also failed to induce T cell apoptosis and offer therapeutic effect for SS and colitis mice. Mechanistic study revealed that Fas-regulated monocyte chemotactic protein 1 (MCP-1) secretion in BMMSCs plays a crucial role in the recruitment of T cells to BMMSCs for FasL-mediated apoptosis. The apoptotic T cells subsequently triggered macrophages to produce high levels of transforming growth factor beta (TGF-β), which led, in turn, to the upregulation of Tregs and, ultimately, immune tolerance for BMMSC-mediated immunotherapies. These data demonstrate a previously unrecognized role of BMMSCs relative to T cell apoptosis through the coupling effect of Fas and FasL in BMMSC-based immunotherapies.

One embodiment of the present invention is directed to the discovery that Fas-regulated monocyte chemotactic protein 1 (MCP-1) secretion in MSCs, preferably BMMSCs, plays a crucial role in the recruitment of T cells to MSCs, preferably BMMSCs, for FasL-mediated apoptosis.

One embodiment of the present invention is directed to the discovery that FasL is required for MSC-, preferably BMMSC-based immune therapies via induction of T cell apoptosis.

One embodiment of the present invention is directed to the discovery that MSCs, preferably BMMSCs, that express Fas and FasL, are unexpectedly more effective than MSCs, preferably BMMSCs, that do not express both proteins for inducing T-cell apoptosis and upregulating Tregs levels.

One embodiment of the present invention is directed to the discovery that the apoptotic T cells subsequently triggered macrophages to produce high levels of transforming growth factor beta (TGF-6), which led, in turn, to the upregulation of Tregs and, ultimately, immune tolerance for BMMSC-mediated immunotherapies.

One embodiment of the present invention is directed to the discovery that treatment of subjects suffering from systemic sclerosis with MSCs, preferably BMMSCs, that express FasL and Fas, and secrete MCP-1 is effective at alleving and/or ameliorating the symptoms of the disease.

One embodiment of the present invention is directed to the discovery that treatment of subjects suffering from colitis with MSCs, preferably BMMSCs, that express FasL and Fas, and secrete MCP-1 is effective at alleving and/or ameliorating the symptoms of the disease.

### BRIEF DESCRIPTION OF THE FIGURES

FIG. 1. BMMSCs induce T cell apoptosis via Fas ligand (FasL). (A) Schema of BMMSC transplantation procedure. 1×10⁶ BMMSCs (n=5), FasL^{-/-} gldBMMSCs (n=4) or FasL-transfected gldBMMSCs (FasL⁺gldBMMSCs, n4) were infused into C57BL6 mice through the tail vein. All groups were sacrificed at indicated time points for sample collection. Zero hour represented that mice were immediately sacrificed after BMMSC injection. (B-E) BMMSC transplantation (BMMSC) induced transient reduction in the number of CD3⁺ T cells and increased annexinV⁺7AAD⁺ double positive apoptotic CD3⁺ T cells in peripheral blood mononuclear cells (PBMNCs; B, C) and bone marrow mononuclear cells (BMMNCs; D, E) at indicated time points, while FasL^{-/-} BMMSCs from gld mice (gldBMMSCs) failed to reduce CD3⁺ T cells or elevate CD3⁺ T cell apoptosis in peripheral blood (B, C) and bone marrow (D, E). FasL-transfected gldBMMSC transplantation (FasL⁺gldBMMSC) partially rescued the capacity to reduce the number of CD3⁺ T cells and induce CD3⁺ T cell apoptosis in peripheral blood (B, C) and bone marrow (D, E). *P<0.05; **P<0.01; ***P<0.001 vs. gldBMMSC, #P<0.05; ^{###}P<0.001. vs. FasL⁺gldBMMSC, ^{$}P<0.05; ^{$$$}P<0.001 vs. gldBMMSC. (F) When BMMSCs were infused into mice, TUNEL and immunohistochemistry staining showed that TUNEL positive apoptotic cells (brown, white arrow) number in CD3-positive T cells (purple, yellow arrowhead) was higher in the BMMSC-injected group compared to the control group in bone marrow. (G) When BMMSCs were co-cultured with T cells, BMMSC-induced annexinV⁺7AAD⁺ double positive apoptotic T cells were significantly blocked by anti-FasL neutralizing antibody (1 μg/mL) compared to IgG antibody control group. (H) TUNEL and immunohistochemistry staining showed that TUNEL positive apoptotic T cells (brown, white arrow) were observed in CD3 T cells (purple, yellow arrowhead) when co-cultured with BMMSCs in vitro. In the presence of anti-FasL neutralizing antibody (FasL Ab), TUNEL-positive cell percentage was significantly less than the untreated control group. (I) In addition, the number of BMMSC-induced annexinV⁺7AAD⁺ double positive apoptotic T cells was significantly blocked by caspase 3, 8, and 9 inhibitor treatments. The results were representative of three independent experiments. (J) Schematic diagram indicating that BMMSCs induce T cell apoptosis. (*P<0.05; **P<0.01; ***P<0.001. The bar graph represents mean±SD).

FIG. 2. FasL is required for BMMSC-induced T cell apoptosis and upregulation of CD4⁺CD25⁺Foxp3⁺ regulatory T cells (Tregs). (A, B) BMMSC transplantation (BMMSC, n=5) induced a transient reduction in the number of CD3⁺ T cells (A) and elevation of annexinV⁺7AAD⁺ double positive apoptotic CD3⁺ cells (B) in peripheral blood. Transplantation of FasL knockdown BMMSC (FasL siRNA BMMSC, n=3) failed to reduce CD3⁺ T cells (A) or increase the number of CD3⁺ apoptotic T cells (B) in peripheral blood. (C, D) BMMSC transplantation (BMMSC, n=5) showed a transient reduction of CD3⁺ T cells (C) and elevation of annexinV⁺7AAD⁺ double positive apoptotic CD3⁺ T cells (D) in bone marrow. Transplantation of FasL knockdown BMMSCs (FasL siRNA BMMSC, n=3) failed to reduce CD3⁺ T cells (C) or elevate CD3⁺ apoptotic T cells (D) in bone marrow. (E) BMMSC, but not FasL knockdown BMMSC, transplantation significantly upregulated levels of Tregs at 24 and 72 hours after transplantation in C57BL6 mice. (F) BMMSC transplantation resulted in a significant up-regulation of Tregs when compared to the gldBMMSC transplantation group at 24 and 72 hours post-transplantation. FasL-transfected gldBMMSC transplantation (FasL⁺gldBMMSC) partially rescued BMMSC-induced upregulation of Tregs. (G) TGF-β level in peripheral blood was significantly increased in both BMMSC and FasL⁺gldBMMSC groups at 24 hours post-transplantation. FasL^{-/-}gldBMMSC transplantation failed to up-regulate TGF-β level. (H) Apoptotic pan T cells were engulfed by macrophages in vivo. Green indicates T cells, and red indicates CD11b⁺ macrophages. Bar=50 μm. (I) BMMSC transplantation group increased the number of CD11b⁺ cells in peripheral blood when compared to the control group (C57BL6). Depletion of macrophages by clodronate liposome treatment showed the effectiveness in reducing CD11b⁺ cells in the BMMSC transplantation group (BMMSC+clodronate), as assessed by flow cytometric analysis. (J) TGF-β level was significantly increased in peripheral blood after BMMSC transplantation. Clodronate liposome treatment blocked BMMSC-induced up regulation of TGF-β (BMMSC+clodronate). (K) BMMSC transplantation upregulated the level of Tregs in peripheral blood compared to the control group (C57BL6). Clodronate liposome treatment inhibited BMMSC-induced Treg upregulation (BMMSC+clodronate). (L) Schematic diagram indicating that BMMSC-induced T cell apoptosis resulted in immune tolerance as evidenced by up-regulation of Tregs. The results were representative of three independent experiments. (*P<0.05, **P<0.01, ***P<0.001. The bar graph represents mean±SD).

FIG. 3. FasL is required for BMMSC-mediated amelioration of systemic sclerosis (SS) phenotypes. (A) Schema showing how BMMSC transplantation ameliorates SS phenotype. (B, C) BMMSC transplantation (n=6) showed a significantly reduced number of CD3⁺ T cells (B) and increased number of annexinV⁺7AAD⁺ double positive apoptotic CD3⁺ T cells (C) in SS mice as assessed by flow cytometric analysis. However, FasL^{-/-} gldBMMSC (n=6) failed to reduce the number of CD3⁺ T cells (B) or elevate the number of apoptotic CD3⁺ T cells (C). (D-F) Tsk/⁺ SS mice showed elevated levels of antinuclear antibody (ANA, D) and anti-double strand DNA antibodies IgG (E) and IgM (F) when compared to control C57BL6 mice. BMMSC transplantation reduced the levels of ANA (D) and anti-double strand DNA antibodies IgG (E) and IgM (F). In contrast, FasL^{-/-} gldBMMSC transplantation failed to reduce the levels of antinuclear antibody (ANA, D) or anti-double strand DNA IgG (E) and IgM (F) antibodies. (G) Creatinine level in serum was significantly increased in Tsk/+ mice. After BMMSC transplantation, creatinine level was significantly decreased to the level observed in C57BL6 mice. However, gldBMMSC transplantation failed to reduce the creatinine level. (H) The concentration of urine protein was significantly increased in Tsk/⁺ mice. BMMSC transplantation reduced urine protein to the control level. gldBMMSC transplantation failed to reduce urine protein levels in Tsk/⁺ mice. (I) Treg level was significantly decreased in Tsk/⁺ mice compared to C57BL6 mice. After BMMSC transplantation, Treg levels were significantly elevated, whereas gldBMMSC transplantation failed to increase Treg levels in Tsk/⁺ mice. (J) CD4+IL17⁺ Th17 cells were significantly increased in Tsk/⁺ mice compared to C57BL6 mice. Elevated Th17 level was significantly reduced in the BMMSC transplantation group, while gldBMMSC transplantation failed to reduce the Th17 level in Tsk/⁺ mice. (K) Hyperdermal thickness was significantly increased in Tsk/⁺ mice (Tsk/⁺, n=5) compared to control mice (C57BL6, n=5). BMMSC, but not FasL^{-/-} gldBMMSC, transplantation reduced hyperdermal thickness. (*P<0.05, **P<0.01, ***P<0.001. The bar graph represents mean±SD).

FIG. 4. FasL plays a critical role in BMMSC-mediated immune therapy for Dextran sulfate sodium (DSS)-induced experimental colitis. (A) Schema showing BMMSC transplantation in DSS-induced experimental colitis mice. (B, C) BMMSC transplantation (n=6) showed a significantly reduced number of CD3⁺ T cells at 24 hours post-transplantation (B) and increased number of annexinV⁺7AAD⁺ double positive apoptotic CD3⁺ T cells at 24-72 hours post-transplantation (C) in colitis mice as assessed by flow cytometric analysis. However, FasL^{-/-} gldBMMSC (n=6) failed to reduce the number of CD3⁺ T cells (B) or elevate the number of apoptotic CD3⁺ T cells (C). (D) Colitis mice (colitis, n=5), BMMSC transplanted group, and gldBMMSC showed significantly reduced body weight from 5 to 10 days after DSS induction. The BMMSC transplantation group showed inhibition of body weight loss compared to the colitis and gldBMMSC transplantation groups at 10 days after DSS induction. (E) Disease activity index (DAI) was significantly increased in colitis mice compared to C57BL6 mice from 5 days to 10 days after DSS induction. BMMSC transplantation significantly reduced DAI score, but it was still higher than that observed in C57BL6 mice. FasL^{-/-} gldBMMSC transplantation failed to reduce DAI score at all time points. (F) Treg level was significantly reduced in colitis mice compared to C57BL6 mice at 7 days after DSS induction. BMMSC, but not FasL^{-/-}gldBMMSC, transplantation upregulated the Treg levels in colitis mice. (G) Th17 cell level was significantly elevated in colitis mice compared to C57BL6 mice at 7 days after DSS induction. BMMSC, but not FasL^{-/-}gldBMMSC, transplantation reduced the levels of Th17 cells in colitis mice from 7 to 10 days after DSS induction. (H) Hematoxylin and eosin staining showed the infiltration of inflammatory cells (blue arrows) in colon with destruction of epithelial layer (yellow triangles) in colitis mice. BMMSC, but not FasL^{-/-}gldBMMSC, transplantation rescued disease phenotype in colon and reduced histological activity index. (I) Schematic diagram of BMMSC transplantation for immunotherapies. (Bar=200 μm; *P<0.05, **P<0.01, ***P<0.001. The bar graph represents mean±SD).

FIG. 5. Fas plays an essential role in BMMSC-mediated CD3⁺ T cell apoptosis and up-regulation of Tregs via regulating monocyte chemotactic protein 1 (MCP-1) secretion. (AD) BMMSC transplantation (BMMSC) induced transient reduction in the number of CD3⁺ T cells and increase in the number of annexinV⁺7AAD⁺ double positive apoptotic CD3⁺ T cells in peripheral blood mononuclear cells (PBMNCs; A, B) and bone marrow mononuclear cells (BMMNCs, n=5; C, D) at indicated time points, while Fas^{-/-} BMMSC from lpr mice (lprBMMSC, n=5) failed to reduce the number of CD3⁺ T cells or increase the number of CD3⁺ apoptotic T cells in peripheral blood (A, B) and bone marrow (C, D). (E, F) lprBMMSC transplantation failed to elevate Treg levels (E) and TGF-β (F) in C57BL6 mice compared to the BMMSC transplantation group at indicated time points. (G) lprBMMSC induced activated T cell apoptosis in a BMMSCT cell in vitro co-cultured system, which was blocked by anti-FasL neutralizing antibody (1 μg/mL). (H-K) Activated T cells (green) migrate to BMMSCs (red) in a transwell co-culture system (H). lprBMMSCs showed a significantly reduced capacity to induce activated T cell migration (I), which could be partially rescued by overexpression of MCP-1 (J) and totally rescued by overexpression of Fas (K) in lprBMMSCs. The results were representative of three independent experiments. (L) Quantitative RT-PCR analysis showed no significant difference between BMMSC and lprBMMSC in terms of MCP-1 expression level. However, overexpression of MCP-1 and Fas in lprBMMSC significantly elevated gene expression level of MCP-1. (M) Western blot showed that lprBMMSCs express a higher cytoplasm level of MCP-1 than BMMSC. Overexpression of Fas in lprBMMSC reduced the expression level of MCP-1 in cytoplasm. (N) ELISA analysis showed that MCP-1 secretion in culture supernatant was significantly reduced in lprBMMSCs compared to BMMSC. Overexpression of MCP-1 and Fas in lprBMMSCs significantly elevated MCP-1 secretion in culture supernatant. (O) ELISA data showed that knockdown Fas expression using siRNA resulted in reduction of MCP-1 level in culture medium compared to control siRNA group. (P-Q) Fas siRNA-treated BMMSCs (Q) showed reduced T cell migration in transwell co-culture system compared to control siRNA group (P). (*P<0.05, **P<0.01, ***P<0.001. The bar graph represents mean±SD).

FIG. 6. MCP-1 plays an important role in T cell recruitment. (A) MCP-1^{-/-}BMMSC transplantation showed a slightly reduced number of CD3⁺ T cells in peripheral blood, but the level of reduction was significantly less than that of the BMMSC transplantation group. (B) AnnexinV⁺7AAD⁺ double positive apoptotic CD3⁺ T cell percentage was slightly increased in the MCP-1^{-/-} BMMSC transplant group. (C) Treg level was slightly increased in the MCP-1^{-/-} BMMSC-transplanted group at 72 hours post-transplantation, but significantly lower than the BMMSC transplantation group. (D) TGF-β level in serum was slightly increased in the MCP-1^{-/-} BMMSC-transplanted group at 72 hours after transplantation compared to 0 hour, but the elevation level was lower than the BMMSC transplantation group. (E/F) When T cells were stimulated with CD3 and CD28 antibody and co-cultured with BMMSC or MCP-1^{-/-} BMMSC in a transwell culture system, the number of migrated T cells was significantly higher in the BMMSC group than the MCP-1^{-/-} BMMSC group. (G) Schematic diagram showing the mechanism of BMMSC-induced immunotherapies. **P<0.01, ***P<0.005, The graph bar represents mean±SD.

FIG. 7. Allogenic MSC transplantation induces CD3⁺ T cell apoptosis and Treg up-regulation in patients with systemic sclerosis (SS). (A) Schema of MSC transplantation in SS patients. (B) Flow cytometric analysis showed reduced number of CD3⁺ T cells from 2 to 72 hours post-transplantation. (C) AnnexinV⁺-positive apoptotic CD3⁺ T cell percentage was significantly increased at 6 hours after MSC transplantation. (D) Flow cytometric analysis showed reduced number of CD4⁺ T cells from 2 to 72 hours post-transplantation. (E) Treg levels in peripheral blood were significantly increased at 72 hours after allogenic MSC transplantation. (F) Serum level of TGFβ was significantly increased in MSC transplantation group at 72 hours post-transplantation. (G, H) Modified Rodnan. Skin Score (MRSS, G) and Health assessment Questionnaire disease activity index (HAQ-DI) (H) were significantly reduced after allogenic MSC transplantation. (I) Representative images of skin ulcers prior to MSC transplantation (pre-MSC) and at 6 months post-transplantation (post-MSC). (J) The reduced ANA level was maintained at 12 months after MSC transplantation. (K) Real-time PCR analysis showed significantly decreased FasL expression in SS patient MSCs (SSMSC) compared to MSC from healthy donor (MSC). (L) SSMSC showed a significantly decreased capacity to induce T cell apoptosis compared to normal MSC in vitro. (M) SSMSC showed a reduced expression of Fas by real-time PCR analysis. (N) MCP-1 secretion level in SSMSC was significantly lower than that in MSC culture supernatant. (*P<0.05, **P<0.01, ***P<0.005; The bar graph represents mean±SD).

FIG. 8. Fas Ligand (FasL) plays an important role in BMMSC-based immunotherapy. (A, B) Western blot analysis showed that mouse BMMSC (mBMMSC) and human BMMSC (hBMMSC) express FasL. CD8⁺ T cells were used as positive control. (C) Immunocytostaining showed that mBMMSC co-expressed FasL (green: middle column) with mesenchymal stem cell surface marker CD73 (red; upper row) or CD90 (red; lower row). (Bar=50̂m). (D) Western blot showed that T cells which were activated by anti CD3 antibody (3 jig/mL) and anti CD28 antibody (2 jig/mL) treatment expressed a higher level of Fas than nave T cells. (E) BMMSC transplantation induced a transient reduction in CD4⁺ and CD8⁺ T cell number in peripheral blood. (F) The percentage of AnnexinV⁺7AAD⁺ double positive apoptotic cells was elevated in both CD4⁺ and CD8⁺ T cells after BMMSC transplantation (**P<0.01, ***P<0.005, vs. 0 h after BMMSC transplantation in CD4⁺ T cell group, ##P<0.01, ###P<0.005 vs. 0 h after BMMSC transplantation in CD8⁺ T cell group. The bar graph represents mean±SD). (G) Schema of BMMSC and anti-Fas Ligand neutralizing antibody (FasLnAb) transplantation in C57BL6 mice. (H, I) BMMSC transplantation, along with FasLnAb injection, showed a significant blockage of BMMSC-induced reduction of CD3⁺ T cell number (H) and elevation of apoptotic CD3⁺ T cells (I) in peripheral blood. (J, K) BMMSC transplantation, along with FasLnAb injection, failed to reduce the number of CD3⁺ T cells (J) and induce CD3⁺ T cell apoptosis (K) in bone marrow. (L) BMMSC transplantation, along with FasLnAb injection, showed lower level of Tregs compared to the BMMSC transplantation group at 72 hours post-transplantation in peripheral blood. (M) BMMSC transplantation, along with FasLnAb injection, showed significant inhibition of BMMSC-induced reduction of Th17 cells in peripheral blood. (N) Flow cytometric analysis showed that transfection of FasL into gldBMMSC could significantly elevate the expression level of FasL. (O) BMMSC transplantation showed downregulated levels of Th17 cells from 6 to 72 hours posttransplantation, while gldBMMSC failed to reduce the number of Th17 cells in peripheral blood. (P, Q) BMMSC transplantation significantly reduced the number of CD3⁺ T cells (P) and induced CD3⁺ T cell apoptosis (Q) at 1.5 hours and 6 hours post-transplantation in spleen. (R, S) BMMSC transplantation induced a transient reduction of the number of CD3⁺ T cells (R) and elevation of apoptotic CD3⁺ T cells (5) in Lymph node. (T) Schema of BMMSC transplantation in OT1TCRTG mice. (U, V) BMMSC transplantation showed upregulation of CD4⁺ T cell apoptosis in peripheral blood (U) and bone marrow (V). (W, X) BMMSC transplantation showed no upregulation of CD8⁺ T cell apoptosis in peripheral blood (W) and bone marrow (X). (Y) BMMSC transplantation in OT1TCRTG mice showed upregulation of Tregs at 24 hours and 72 hours post-transplantation. (Z) BMMSC transplantation in OT1TCRTG mice showed reduction of Th17 cell level from 24 hours to 72 hours post-transplantation in peripheral blood. (AA) CD8⁺ T cell in OT1TCRTG mice showed no alteration in BMMSC transplantation group. (*P<0.05, **P<0.01, ***P<0.005. The bar graph represents mean±SD).

FIG. 9. Immunomodulation property of syngenic mouse BMMSC and human BMMSC transplantation. (A) Schema of syngenic and allogenic BMMSC transplantation in C57BL6 mice. (B, C) Both syngenic and allogenic BMMSC transplantation showed similar effect in reducing the number of CD3⁺ T cells (B) and inducing CD3⁺ T cell apoptosis (C) in peripheral blood. (D, E) Both syngenic and allogenic BMMSC transplantation reduced the number of CD3⁺ T cells (D) and induced CD3⁺ T cell apoptosis (E) in bone marrow. (F, G) Both syngenic and allogenic BMMSC transplantation upregulated levels of Tregs (F) and downregulated levels of Th17 cells (G) in peripheral blood, while allogenic BMMSC transplantation showed a more significant reduction of Th17 cells compared to syngenic BMMSCs at 24 and 72 hours post-transplantation. (H) Flow cytometric analysis showed culture expanded human BMMSCs (hBMMSCs) express the stem cell markers CD73, CD90, CD105, CD146, and Stro1, but they are negative for the hematopoietic markers CD34 and CD45. Isotopic IgGs were used as a negative control. (I) Schema of human bone marrow mesenchymal stem cell (hBMMSC) transplantation in C57BL6 mice. (J, K) hMSC infusion induced CD3⁺ T cell apoptosis in peripheral blood (J) and bone marrow (K) in C57BL6 mice. (L, M) hMSC infusion induced upregulation of Tregs (L) and downregulation of Th17 cells (M) in peripheral blood. (*P<0.05, **P<0.01, ***P<0.005. The bar graph represents mean±SD).

FIG. 10. Apoptosis of transplanted BMMSCs. (A) Western blot showed efficacy of FasL siRNA. (B) Immunofluorescent analysis showed that Annexin⁺/7AAD⁺ double positive apoptotic cells, including transplanted GFP⁺BMMSC (white arrowhead) and recipient cells (orange arrow) at 6 hours post-transplantation in peripheral blood (upper row) and bone marrow (lower row). Bar=50 Vm. (C-F) Carboxyfluorescein diacetate N-succinimidyl ester (CFSE)-labeled control BMMSCs, FasL^{-/-} gldBMMSCs and FasL siRNA BMMSCs were transplanted into C57BL6 mice. Peripheral blood and bone marrow samples were collected at indicated time points for cytometric analysis. The number of CFSE-positive transplanted BMMSCs reached a peak at 1.5 hours post-transplantation in peripheral blood (C) and bone marrow (D) and then reduced to undetectable level at 24 hours post-transplantation. The number of AnnexinV⁺7AAD⁺ double positive apoptotic BMMSCs reached a peak at 6 hours post-transplantation in peripheral blood (E) and bone marrow (F) and then reduced to an undetectable level at 24 hours posttransplantation. (The bar graph represents mean±SD)

FIG. 11. FasL is required for BMMSC-mediated amelioration of skin phenotype in systemic sclerosis (SS) mice. (A) Systemic sclerosis mouse model (Tsk/⁺) showed tight skin phenotype compared to control C57BL6 mice. BMMSC, but not FasL^{-/-} gldBMMSC, transplantation significantly improved skin phenotype in terms of grabbed skin distance. (B) BMMSC transplantation maintained spleen Treg level as observed in control mice at 2 month post-transplantation. (*P<0.05, **P<0.01. The bar graph represents mean±SD).

FIG. 12. Tregs are required in BMMSC-mediated immune therapy for DSS-induced experimental colitis. (A) Schema of BMMSC transplantation with blockage of Treg using anti-CD25 antibody in DSS-induced colitis mice. (B) Colitis mice (colitis, n=5), BMMSC-treated colitis mice (n=6), and BMMSC-treated colitis mice with anti-CD25 antibody injection (BMMSC+antiCD25ab, n=5) showed reduced body weight from 5 to 10 days after DSS induction. BMMSC transplantation, but not BMMSC transplantation along with anti CD25ab injection, could partially inhibit colitis-induced body weight loss at 10 days after DSS induction. (C) Disease Activity Index (DAI) was significantly increased in colitis mice compared to C57BL6 mice from 5 to 10 days after DSS induction. BMMSC transplantation significantly reduced the DAI score compared to colitis model, but it was still higher than that observed in C57BL6 mice. The BMMSC+antiCD25ab group failed to reduce the DAI score at all observed time points. (D) Treg level was significantly reduced in colitis mice compared to C57BL6 mice at 7 days after DSS induction. The BMMSC transplantation group showed upregulation of Treg levels in colitis mice. The BMMSC+antiCD25ab group showed reduced Treg level at all time points. (E) Th17 cell level was significantly elevated in colitis mice compared to C57BL6 mice at 7 days after DSS induction. The BMMSC transplantation reduced the levels of Th17 cells in colitis mice from 7 to 10 days after DSS induction. The BMMSC+antiCD25ab group showed lower level of Th17 cells compared to colitis group, but still higher than the BMMSC group at 10 days post-DDS induction. (F) Hematoxylin and eosin staining showed the infiltration of inflammatory cells (blue arrows) in colon with destruction of epithelial layer (yellow triangles) in colitis mice. The BMMSC transplantation group showed rescued disease phenotype in colon and histological activity index, while the BMMSC+antiCD25ab group failed to reduce disease phenotype at 10 days after DSS induction. (Bar=200̂m; *P<0.05, **P<0.01, ***P<0.001. The bar graph represents mean±SD)

FIG. 13. Fas is required for ameliorating disease phenotype in induced experimental colitis and systemic sclerosis (SS). (A) Western blot analysis showed that mouse BMMSCs express Fas. CD8⁺ T cells were used as a positive control. (B) Schema of BMMSC transplantation in experimental colitis mice. (C) lprBMMSC transplantation failed to inhibit body weight loss in colitis mice. (D) Increased disease activity index in colitis mice was not reduced in the lprBMMSC transplantation group. (E) Histological analysis of colon showed no remarkable difference between experimental colitis mice and lprBMMSC transplantation group. Bar=200 nm. (F) IprBMMSC transplantation failed to upregulate Treg level in experimental colitis mice. (G) Increased Th17 level in experimental colitis mice was not reduced in the lprBMMSC transplantation group. (H) Schema of BMMSC transplantation in Tsk/⁺ mice. (I) Increased ANA level in SS (Tsk/⁺) mice was not reduced in the lprBMMSC transplantation group. (J, K) The levels of Anti-dsDNA were not reduced in lprBMMSC treated Tsk/⁺ mice (IgG: J, IgM; K). (L) Increased creatinine level in TAP/⁺ mice was not reduced in the lprBMMSC transplantation group. (M) lprBMMSC failed to reduce urine protein level in Tsk/⁺ mice. (N) Bent vertebra and skin tightness, as indicated by grabbed distance in Tsk/⁺ mice, were not improved in the lprBMMSC transplantation group. (O) The reduced Treg level in Tsk/⁺ mice was not upregulated in lprBMMSC transplantation group. (P) lprBMMSC transplantation failed to reduce Th17 level in Tsk/⁺ mice. (Q) lprBMMSC transplantation failed to reduce hypodermal thickness in Tsk/⁺ mice. (R) Western blot analysis showed that Fas^{-/-}VprBMMSCs express FasL at the same level as observed in BMMSCs. (S) Cytokine array analysis showed that BMMSCs express a higher level of MCP-1 than lprBMMSCs in the culture supernatant. After Fas overexpression in Fas^{-/-}lprBMMSC (Fas⁺lprBMMSC) by cDNA transfection, the secretion level of multiple cytokines/chemokines was restored to the level observed in BMMSCs. (T) Western blot analysis showed efficacy of Fas siRNA in BMMSCs. (U) Flow cytometric analysis showed that transfection of Fas into lprBMMSCs could significantly elevated the expression level of Fas. (V-W) ELISA analysis showed that Fas^{-/-}VprBMMSCs and Fas knockdown BMMSCs (Fas siRNA BMMSC) had a significantly reduced level of CXCL-10 (V) and TIMP-1 (W) in the culture supernatant compared to BMMSCs or control siRNA group. (X) BMMSC transplantation showed downregulated levels of Th17 cells from 6 to 72 hours post-transplantation, while lprBMMSCs failed to reduce the number of Th17 cells in peripheral blood. (Y) Schema of Fas knockdown BMMSC transplantation in C57BL6 mice. (Z, AA) Fas knockdown BMMSCs using siRNA (Fas siRNA BMMSC) showed a significantly reduced capacity to reduce the number of CD3⁺ T cells (Z) and induce CD3⁺ T cell apoptosis (AA) in peripheral blood. (BB, CC) Fas siRNA BMMSCs showed reduced capacity to reduce the number of CD3⁺ T cells (BB) and induce CD3⁺ T cell apoptosis (CC) when compared to the BMMSC transplantation group in bone marrow. (DD) Fas siRNA BMMSCs failed to upregulate Tregs compared to the BMMSC group in peripheral blood. (EE) Fas siRNA BMMSC failed to significantly reduce Th17 cell compared to BMMSC group in peripheral blood. (*P<0.05, **P<0.01, ***P<0.005. The bar graph represents mean±SD).

FIG. 14. Fas and MCP-1 regulate BMMSC-mediated B cell, NK cell, and immature dendritic cell (iDC) migration in vitro. (A-C) When B cells, NK cells, and iDCs were co-cultured with BMMSCs, FaŝlprBMIVISCs, Fas knockdown BMMSCs using siRNA (Fas siRNA BMMSC), or MCP-1′″″ BMMSCs in a transwell culture system, the number of migrated B cells (A), NK cells (B), and iDCs (C) was significantly higher in the BMMSC group. (**P<0.01. Bar=100̂m. The bar graph represents mean±SD).

### DETAILED DESCRIPTION OF THE INVENTION

Abbreviations:

MSCs: mesenchymal stem cells

BMMSCs: bone marrow mesenchymal stem cells;

BMMSCT: bone marrow mesenchymal stem cell transplantation;

FasL: Fas ligand;

hMSCs: human mesenchymal stem cells;

hBMMSCs: human bone marrow mesenchymal stem cells;

MCP-1: Monocyte chemoattractant protein-1

SS: systemic sclerosis;

Tregs: CD4⁺CD25⁺Foxp3⁺ regulatory T cells.

### DEFINITIONS

As used herein, “allogenic” means having a different genetic makeup, such as from two different species or from two unrelated subjects of the same species.

An “effective amount” of a composition as used in the methods of the present invention is an amount sufficient to carry out a specifically stated purpose. An “effective amount” may be determined empirically and in a routine manner in relation to the stated purpose.

As used herein, “expression” or “expressing” includes the process by which polynucleotides are transcribed into mRNA and translated into peptides, polypeptides, or proteins. “Expression” can include natural expression and overexpression. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgamo sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general. In a preferred embodiment, MSCs express Fas at a level greater than the level of Fas expression exhibited by Fas^{-/-} lprBMMSC cells and express FasL at a level greater than the level of FasL expression exhibited by FasL^{-/-} gldBMMSC cells, as measured by techniques known in the art.

The terms “expression vector” or “vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

An “isolated” and “purified” MSC population is a population of MSCs that is found in a condition apart from its native environment and apart from other constituents in its native environment, such as blood and animal tissue. In its preferred form, an isolated and purified MSC population is enriched for MSCs that a) express Fas, b) express FasL, and c) secrete MCP-1. In a preferred form, the isolated and purified MSC population is substantially free of cells that are not MSC cells and animal tissue, and more preferably substantially free of other MSCs that do not a) express Fas, b) express FasL, and c) secrete MCP-1. It is preferred to provide the MSC population in a highly purified form, i.e. greater than 50% pure (as a percentage of cells that express Fas, b) express FasL, and c) secrete MCP-1 to the total population of cells), greater than 80% pure, greater than 90% pure, greater than 95% pure, and more preferably greater than 99% pure. Non-limiting examples of methods for isolating and purifying MSCs are provided herein.

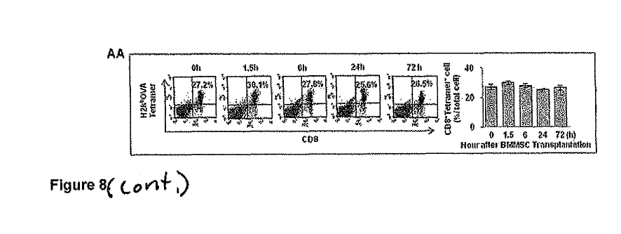

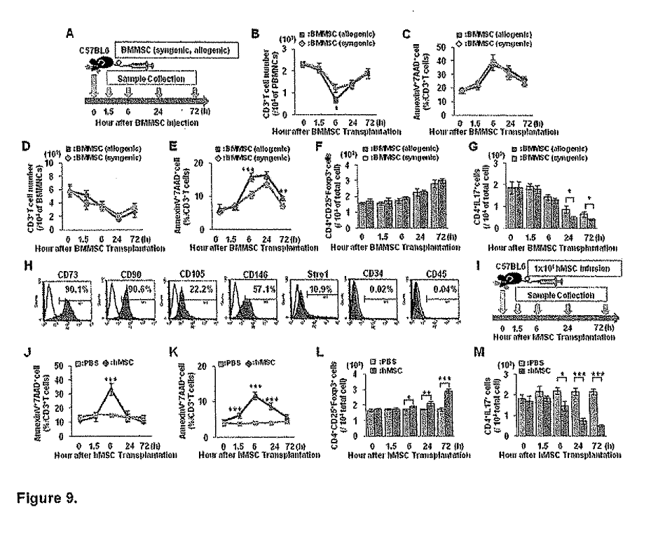

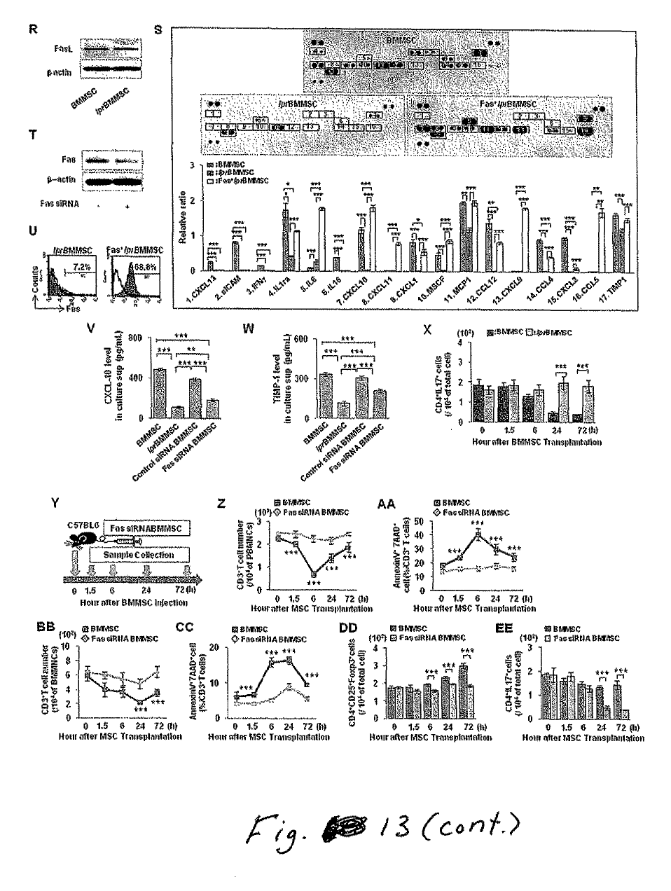

The terms “overexpression” and “overexpressing”, are used in reference to levels of mRNA or protein to indicate a level of expression from a transgenic or artificially induced cell greater than the level of expression from the unmodified and/or uninduced control. With respect to the BMMSCs of the present invention, it is preferable that the level of overexpression of FasL be at least 5-fold higher than the level of expression of FasL exhibited by FasL−/− gldBMMSCs (FIG. 8N). With respect to the BMMSCs of the present invention, it is preferable that the level of overexpression of Fas be at least 5-fold higher than the level of expression of Fas exhibited by Fas−/− lprBMMSCs (FIG. 13U). Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA. Levels of protein are measured using any number of techniques known to those skilled in the art including, but not limited to flow cytometric analysis. As used herein, “syngenic” means having an identical or closely similar genetic makeup, such as from the host or from a familial relative.

The term “upregulating” is used herein to mean increasing, directly or indirectly, the presence or amount of the substance being measured.

Unless otherwise indicated, all terms used herein have the meanings given below, and are generally consistent with same meaning that the terms have to those skilled in the art of the present invention. Practitioners are particularly directed to Alberts et al. (2008) Molecular Biology of the Cell (Fifth Edition (Reference Edition)) Garland Science, Taylor & Francis Group, LLC, for definitions and terms of the art. It is to be understood that this invention is not limited to the particular methodology, protocols, and reagents described, as these may vary.

Any type of isolated mesenchymal stem cells (MSCs) may be suitable for the purposes of this invention. Such mesenchymal cells may be isolated from a variety of organisms. Preferably the MSCs are isolated from murine or human sources. Most preferably, the MSCs are isolated from human sources. The MSCs may be isolated from a variety of tissue types. For example, MSCs may be isolated from bone marrow, umbilical cord tissue, and umbilical cord blood. MSCs may be isolated from a tissue present at the organism's oral cavity. For example, apical papilla stem cells (SCAPs), periodontal ligament stem cells (PDLSCs), and dental pulp stems cells (DPSCs), which are isolated from a tissue present at a human's oral cavity may be used. Such human MSCs are disclosed, for example, in the U.S. patent application publication, No. 20100196854 to Shi et al. entitled “Mesenchymal Stem Cell-Mediated Functional Tooth Regeneration”, which is incorporated by reference herein in the entirety. In one embodiment, human mesenchymal stem cells (hMSCs) may be isolated from human bone marrow.

One embodiment of the invention relates to a method of treating systemic sclerosis in a subject in need thereof comprising administering a therapeutically effective amount of mesenchymal stem cells (MSCs) to the subject, wherein said MSCs a) express Fas, b) express FasL and c) secrete MCP-1.

Preferably, the method comprises administering a composition comprising an isolated and purified population of said MSCs. Preferably, the method comprises administering MSCs that are bone marrow MSCs (BMMSCs), more preferably human BMMSCs.

The MSCs of the present invention may be syngenic or allogenic, and preferably are allogenic. Preferably from 1×10³ to 1×10⁷ cells per kg body weight of said MSCs is administered. More preferably, from 1×10⁵ to 1×10⁷ cells per kg body weight of said MSCs are administered. Preferably, administration of said MSCs is by infusion or by transplantation.

Another embodiment of the present invention realtes to a method of treating systemic sclerosis in a subject in need thereof comprising administering a composition comprising a therapeutically effective amount of an isolated and purified population of allogenic hBMMSCs to the subject, wherein said hBMMSCs a) express Fas, b) express FasL and c) secrete MCP-1.

Preferably, from 1×10³ to 1×10⁷ cells per kg body weight of said hBMMSCs are administered. More preferably, from 1×10⁵ to 1×10⁷ cells per kg body weight of said hBMMSCs are administered. Preferably be administration is by infusion or by transplantation.

Another embodiment of the present invention relates to a method of treating colitis in a subject in need thereof comprising administering a therapeutically effective amount of MSCs to the subject, wherein said MSCs a) express Fas, b) express FasL and c) secrete MCP-1.

Preferably, the method comprises administering a composition comprising an isolated and purified population of said MSCs. Preferably the MSCs are BMMSCs, and more preferably the MSCs are human BMMSCs. Preferably, from 1×10³ to 1×10⁷ cells per kg body weight of said hBMMSCs are administered. More preferably, from 1×10⁵ to 1×10⁷ cells per kg body weight of said hBMMSCs are administered. Preferably, the BMMSCs are administered by infusion or by transplantation.

Another aspect of the present invention relates to an isolated and purified population of MSCs, wherein said MSCs a) express Fas, b) express FasL and c) secrete MCP-1. Preferably the MSCs are BMMSCs, more preferably human BMMSCs.

Another aspect of the present invention relates an isolated and purified population of MSCs, wherein said MSCs a) express Fas, b) express FasL and c) secrete MCP-1, that have been transfected with a vector comprising a gene for human. FasL operably linked to a promoter, and wherein FasL is overexpressed from said vector. Another aspect of the present invention relates an isolated and purified population of MSCs, wherein said MSCs a) express Fas, b) express FasL and c) secrete MCP-1, that have been transfected with a vector comprising a gene for human Fas operably linked to a promoter, and wherein Fas is overexpressed from said vector. The MSCs of the present invention may be transfected with the genes for either FasL or Fas, or both.

Another aspect of the present invention relates to a method of upregulating regulatory T cells (Treg) in a human comprising administering an effective amount of hBMMSCs to the human, wherein said hBMMSCs a) express Fas, b) express FasL, and c) secrete MCP-1. Preferably the human is suffering from systemic sclerosis. Preferably the human is suffering from colitis.

Preferably, the method of upregulating regulatory T cells (Treg) is practiced by administering allogenic hBMMSCs. Preferably, from 1×10³ to 1×10⁷ cells per kg body weight of said hBMMSCs are administered. More preferably, from 1×10⁵ to 1×10⁷ cells per kg body weight of said hBMMSCs are administered. Preferably, the BMMSCs are administered by infusion or by transplantation.

Preferably, administration according to the present method of upregulating regulatory T cells (Treg) causes a reduction in the number of CD4+ T cells and a corresponding increase in the number of apoptotic CD4+ T cells. The method preferably causes a reduction in the number of CD8+ T cells and a corresponding increase in the number of apoptotic CD8+ T cells. Preferably, the method causes a reduction in the number of CD3+ T cells and a corresponding increase in the number of apoptotic CD3+ T cells. Preferably the method causes a reduction in the number of two or more, or all, of said. T cell sub-populations, together with a corresponding increase in the same two or more, or all, of said T-cell sub-populations.

More preferably, the method of upregulating regulatory T cells (Treg) of the present invention results in levels of regulatory T cells in peripheral blood that are significantly upregulated about 72 hours after administration.

Another embodiment of the invention relates to a method of producing immune tolerance to immunotherapies in a subject in need thereof comprising administering an effective amount of hBMMSCs, wherein said hBMMSCs a) express Fas, b) express FasL, and c) secrete MCP-1, and wherein said administration causes an upregulation in the level of regulatory T cells in the peripheral blood of the subject.

Another embodiment of the invention relates to a pharmaceutical composition comprising an isolated and purified population of MSCs, wherein said MSCs a) express Fas, b) express FasL, and c) secrete MCP-1, dispersed in a pharmaceutically acceptable carrier.

Herein is provided experimental evidence that MSC-induced in vivo activated T cell apoptosis via Fas/FasL pathway plays a critical role in inducing immune tolerance and thus offering a novel therapeutic option for systemic sclerosis and inductive experimental colitis mice.

The FasL/Fas-mediated cell death pathway represents typical apoptotic signaling in many cell types (Hohlbaum et al., 2000; Pluchino et al., 2005; Andersen et al., 2006; Zhang et al., 2008). MSCs derived from bone marrow (BMMSCs) express FasL and induce tumor cell apoptosis in vitro (Mazar et al., 2009). However, it is unknown that whether BMMSCs induce T cell apoptosis via Fas/FasL pathway leading to immune tolerance. We transplanted BMMSCs into C57BL6 mice and demonstrated that BMMSCs expressing FasL, but not FasL-deficient BMMSCs, induced transient T cell apoptosis. Furthermore, we found that reduced number of T cells occurred in multiple organs, including peripheral blood, bone marrow, spleen, and lymph node. It appears that alteration of T cell number, owing to T cell redistribution, is not supported by the experimental evidence. Since CD3 antibody-induced T cell apoptosis resulted in immune tolerance (Chatenoud et al., 1994 and 1997), we confirm here that BMMSC-induced T cell apoptosis upregulates Tregs via high levels of macrophage-released TGF-β (Kleinclauss et al., 2006; Perruche et al., 2008). Although transplanted FasL^{-/-} gldBMMSCs and FasL knockdown BMMSCs undergo apoptosis in vivo, they failed to induce upregulation of Tregs. This evidence further confirms that T cell apoptosis, but not transplanted BMMSCs, is required for inductive up-regulation of Tregs (Perruche et al., 2008). BMMSC-induced CD3⁺ T cell apoptosis reaches a peak at 24 hours post-transplantation in a chronic inflammatory disease tight-skin (Tsk/+) mouse model and at 6 hours post-transplantation in an acute inflammatory disease experimental colitis mouse model. Therefore, BMMSC-induced T cell apoptosis may be regulated by the condition of recipient immune system.

Despite the expression of functional FasL by Fas^{-/-} lprBMMSCs, they failed to induce T cell apoptosis and upregulate Tregs in vivo. Mechanistically, Fas controls chemoattractant cytokine MCP-1 secretion in BMMSCs. Decreased MCP-1 secretion from lprBMMSC results in the failure to recruit activated T cells to BMMSCs (Carr et al., 1994; Xu et al., 1996) and, hence, infusion of Fas^{-/-} lprBMMSCs failed to induce T cell apoptosis in viva. However, when lprBMMSCs were directly co-cultured with CD3⁺ T cells, they could induce T cell apoptosis, suggesting that lprBMMSC may not able to initiate cell-cell contact with T cells in viva. Moreover, Fas^{-/-} lprBMMSCs show a higher cytoplasm level of MCP-1 than control BMMSCs, suggesting that Fas regulates MCP-1 secretion, but not MCP-1 production. When MCP-1^{-/-} BMMSCs were systemically transplanted into C57BL6 mice, CD3⁺ T cell apoptosis and Treg upregulation were significantly reduced compared to MCP-1-secreting BMMSC group, suggesting that MCP-1 is one of the factors regulating MSC-based immune tolerance. It was reported that BMMSCs could inhibit CD4/Th17 T cells with MCP-1 paracrine conversion from agonist to antagonist (Rafei et al., 2009). Here we showed that MCP-1 helped to recruit T cells to up-regulate Tregs. It was reported that BMMSC transplantation induced immune tolerance in Fas null lpr mice via inducing delayed T cell apoptosis, upregulated Tregs, and downregulated Th17 cells (Sun et al., 2009), suggesting that BMMSCs are capable of inducing T cell apoptosis and immune tolerance through a non-Fas/FasL pathway. When the Fas/FasL pathway is blocked, BMMSCs could interact with T cells via an alternative pathway to cause T cell apoptosis.

Significantly, our primary clinical investigation showed that Fas- and FasL-expressing MSC infusion induced CD3⁺ T cell apoptosis and Treg upregulation in allogenic MSC-infused SS patients. In our 1-12 month follow-up period, we did not find any clinical sign of side effects, including cardiovascular and pulmonary insufficiencies, infection, malignancy, or metabolic disturbances, suggesting the safety of the MSC therapy in SS patients. The therapeutic effects of allogenic MSC transplantation were significant as shown by the reduction of MRSS, HAQDI, in addition to improved quality of life. Furthermore, we demonstrated that MSC transplantation dramatically improved treatment-refractory skin ulcers.

Thus, we have uncovered a previously unrecognized BMMSC-mediated therapeutic mechanism by which BMMSCs use Fas to regulate MCP-1 secretion for T cell recruitment and subsequently use FasL to induce T cell apoptosis. Macrophages subsequently take the debris of apoptotic T cells to release a high level of TGF-β, leading to upregulation of Tregs and, ultimately, immune tolerance for immunotherapies. Collaborative execution of therapeutic effect between Fas and FasL may therefore represent a new functional role of receptor/ligand in cell-based therapies.

In the methods described herein, the effective amount of the MSCs, can range from the maximum number of cells that is safely received by the subject to the minimum number of cells necessary for to achieve the intended effect. Preferably, the effective amount is from 1×10⁸ cells/kg body weight to 1×10⁷ cells/kg body weight, more preferably from 1×10⁵ cells/kg body weight to 1×10⁷ cells/kg body weight. More preferably, the effective amount is about 1×10⁶ cells/kg body weight.

The effective amount of the MSCs can be suspended in a pharmaceutically acceptable carrier or excipient. Such a carrier may include but is not limited to a suitable culture medium plus 1% serum albumin, saline, buffered saline, dextrose, water, and combinations thereof. The formulation should suit the mode of administration.

In a preferred embodiment, the MSC preparation or composition is formulated in accordance with routine procedures as a pharmaceutical composition adapted for systemic administration to human beings. Typically, compositions for systemic administration are solutions in sterile isotonic aqueous buffer. When the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

A variety of means for administering cells to subjects will be apparent to those of skill in the art. Such methods include may include systemic administration or injection of the cells into a target site in a subject. Cells may be inserted into a delivery device which facilitates introduction by injection or implantation into the subjects. Such delivery devices may include tubes, e.g., catheters, for injecting cells and fluids into the body of a recipient subject. In a preferred embodiment, the tubes additionally have a needle, e.g., a syringe, through which the cells of the invention can be introduced into the subject at a desired location. The cells may be prepared for delivery in a variety of different forms. For example, the cells may be suspended in a solution or gel. Cells may be mixed with a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid, and will often be isotonic. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Modes of administration of the MSCs include but are not limited to systemic intravenous or intra-arterial injection, injection directly into the tissue at the intended site of activity and transplantation. The preparation can be administered by any convenient route, for example by infusion or bolus injection and can be administered together with other biologically active agents. Administration is preferably systemic. It may be advantageous, under certain conditions, to use a site of administration close to or nearest the intended site of activity. Without intending to be bound by mechanism, GMSCs will, when administered, migrate or home to the tissue in response to chemotactic factors produced due to the inflammation or injury.

The following Examples are provided in order to demonstrate and further illustrate certain embodiments and aspects of the present invention and are not to be construed as limiting the scope thereof.

### EXPERIMENTAL METHODS

### Experimental Procedures

Animals and antibodies. Female C57BL6J (BL6), B6CgFbln^{TSK+/+}Pldn^{Pa}/J, C57BL16-Tg(TcraTcrb)1100Mjb/J (OT1TCRTG), B6Smn.C3-Fasl^{gld}/J (BL6 gld), C3MRL-Fas^{lpr}/J (C3H lpr), and B6.129S4-Ccl2^{tm1Rol}/J mice were purchased from the Jackson Lab. gld and lpr strain have spontaneous mutation in FasL (Fasl^{gld}) and Fas (Fas^{lpr}), respectively, with no other spontaneous mutation. Female immunocompromised mice (Beige nude/nude XIDIII) were purchased from Harlan. All animal experiments were performed under the institutionally approved protocols for the use of animal research (USC #10941 and 11327). The antibodies used in this study are described herein.

Isolation and Purification of Human MSCs.

hMSCs may be isolated by using any previously disclosed method. For example, a mesenchymal stem cell isolation method disclosed in a publication to Shi et al. (2003) “Perivascular Niche of Postnatal Mesenchymal Stem Cells in Human Bone Marrow and Dental Pulp” J. Bone Miner. Res., 18(4), 696-704 may be used for this purpose. The entire content of this publication is incorporated herein in the entirety. In one embodiment, hMSCs may be isolated by immunoselection using the antibody, STRO-1, which recognizes an antigen in a tissue comprising hMSCs.

Isolation of Mouse Bone Marrow Mesenchymal Stem Cells (BMMSCs).

The mouse BMMSCs were isolated from femurs and tibias and maintained.

Isolation of CD11b-Positive Cells.

To isolate CD11b-positive phagocytes, mouse splenocytes were isolated and incubated with PE-conjugated anti-CD11b antibody (BD). After 30 min incubation on ice, CD11b-positive cells were sorted out using anti-PE magnetic beads (Miltenyi Biotech) according to manufacturer's instructions.

Flow Cytometry Analysis.

Whole peripheral blood was stained with anti-CD45, anti-CD3, anti-CD4, and CD8a antibodies and treated with BD FACS™ Lysing Solution (BD Bioscience) to get mononuclear cells (MNCs). The apoptotic T cells were detected by staining with CD3 antibody, followed by Annexine-V Apoptosis Detection Kit I (BD Pharmingen). For fluorescent labeling of cells, BMMSCs or T cells were incubated with Carboxyfluorescein diacetate N-succinimidyl ester (CFSE, SIGMA) for 15 min or PKH-26 (Invitrogen) for 5 min, according to manufacturer's instructions. For Foxp3 intercellular staining, T cells were stained with anti-CD4, CD8a, and CD25 antibodies (1 μg each) for 30 min on ice. Next, cells were stained with anti-Foxp3 antibody using Foxp3 staining buffer kit (eBioscience). For IL17 staining, T cells were stained with anti-CD4 antibody and then stained with anti-IL17 antibody using Foxp3 staining buffer kit. All samples were analyzed with FACS^{calibur} (BD Bioscience).

Western Blot Analysis.

20 g of protein were used and SDS-PAGE and Western blotting were performed according to standard procedures. β-actin on the same membrane served as the loading control. Detailed procedures are described in

Real-Time Polymerase Chain Reaction (RT-PCR).

100 ng of total RNA was used for cDNA synthesis and RT-PCR. The gene-specific primer pairs are as follows: Human FasL (GeneBank accession number; NM_{—}000639.1, sense; 5′-CTCTTGAGCAGTCAGCAACAGG-3′, antisense; 5′-ATGGCAGCTGGTGAGTCAGG-3), human Fas (GeneBank accession number; NM_{—}000043.4, antisense;

5′-CAACAACCATGCTGGGCATC-3′, sense;

5′-TGATGTCAGTCACTTGGGCATTAAC-3), and human GAPDH (GeneBank accession number; NM_{—}002046.3, antisense;

5′-GCACCGTCAAGGCTGAGAAC-3′, sense; TGGTGAAGACGCCAGTGGA). Detailed procedures are described in

Co-Culture of T Cells with BMMSCs.

BMMSCs (0.2×10⁶) were seeded on a 24-well culture plate (Corning) and incubated 24 hours. The prestimulated T cells were directly loaded onto BMMSCs and co-cultured for 2 days. In some experiments, anti-Fas ligand neutralizing antibody (BD) or caspase 3, 8 or 9 inhibitors (R&D systems) were added in the co-culture. Apoptotic T cells were detected as described above.

T Cell Migration Assay.

For T cell migration assay, a transwell system was used. PKH26-stained BMMSCs (0.2×108) were seeded on the lower chamber of a 12-well culture plate (Corning) with transwell and incubated 24 hours. The prestimulated T cells with anti-CD3 and Anti-CD28 antibodies for 48 hours were loaded onto upper chamber of transwell and co-cultured for 48 hours and observed under a fluorescent microscope. Green-labeled cell number was counted and normalized by red-labeled number of MSCs in five representative images.

Overexpression of Fas Ligand.

293T cells for lentivirus production were seeded in a 10 cm culture dish (Corning) until 80% confluence. Plasmids with proper proportion, FasL gene expression vector: psPAX:pCMV-VSV-G (all from Addgene)=5:3:2, were mixed in opti-MEM (Invitrogen) with Lipofectamin LTX (Invitrogen) according to the protocol of the manufacturer. EQFP expression plasmid (Addgene) was used as control. The supernatant was collected 24 h and 48 h after transfection and filtered through a 0.45 μm filter to remove cell debris. For infection, the supernatant containing lentivirus was added into target cell culture in the presence of 4 μg/ml polybrene (SIGMA), and the transgene expression was validated by GFP under microscopic observation.

Overexpression of Fas and MCP-1.

To generate Fas and MCP-1 overexpression vectors, a pCMV6-AC-GFP TrueORF mammalian expression vector system (Origene) was used. Fas and MCP-1 cDNA clones generated from C57BL/6J strain mice were purchased from Open Biosystems (Hunteville) and amplified by PCR with Sgf I and Mlu I restriction cutting sites. The PCR products were directly subcloned into pCR-Blunt II-TOPO vector using Zero Blunt® TOPO PCR Cloning Kit (Invitrogene). After sequencing, Fas and MCP-1 cDNAs with SgfI/MluI sites were subcloned into pCMV6-AC-GFP expression vector. All constructs were verified by sequencing before transfection into cells. After construction, lprBMMSCs were transfected with cDNAs using LIPOFECTAMINE PLUS reagent (LIFE TECHNOLOGIES), according to manufacturer's instructions for 48 hours.

Inhibition of Fas and FasL.

Expression levels of Fas and FasL on BMMSCs were knocked down using siRNA transfection according to manufacturer's instructions. Fluorescein conjugated control siRNA was used as control and as a method of evaluating transfection efficacy. All siRNA products were purchased from Santa Cruz.

Allogenic BMMSC Transplantation into Acute Colitis Mice.

Acute colitis was induced by administering 3% (w/v) dextran sulfate sodium (DSS, molecular mass 36,000-50,000 Da; MP Biochemicals) through drinking water, which was fed ad libitum for 10 days (Zhang et al., 2010). Passage one BMMSCs, gldBMMSCs or lprBMMSCs were infused (1×10⁶ cells) into disease model mice (n=6) intravenously at day 3 after feeding DSS water. In control group, mice received PBS (n=6). All mice were harvested at day 10 after feeding DSS water and analyzed. Induced colitis was evaluated as previously described (Alex et al., 2009).

Allogenic BMMSC Transplantation into Systemic Sclerosis (SS) Mice.

Passage one BMMSCs, gldBMMSCs or lprBMMSCs were infused (1×10⁶ cells) into SS mice intravenously at 8 weeks of age (n=6). In control group, SS mice received PBS (n=5). All mice were sacrificed at 12 weeks of age for further analysis. The protein concentration in urine was measured using Bio-Rad Protein Assay (Bio-Rad).

Allogenic MSC Transplantation into Systemic Sclerosis (SS) Patients.

MSCs from umbilical cord were sorted out and expanded, following a previous report (Liang et al., 2009). Expanded MSCs were intravenously infused into the SS recipients (1×10⁶/kg body weight). The trial was conducted in compliance with current Good Clinical Practice standards and in accordance with the principles set forth under the Declaration of Helsinki, 1989. This protocol was approved by the IRB of the Drum Tower Hospital of Nanjing, University Medical School, China. Informed consent was obtained from each patient.

Statistical Analysis.

Student's t-test was used to analyze statistical difference. The p values less than 0.05 were considered significant.

Antibodies.

Anti-mouse-CD4-PerCP, CD8-FITC, CD25-APC, CD11b-PE, CD34-FITC, CD45-APC, CD73-PE, CD90.2-PE, CD105-PE, CD117-PE, Sca-1-PE, CD3s, CD28, anti-human-CD73-PE, CD90-PE, CD105-PE, CD146-PE, CD34-PE and CD45-PE antibodies were purchased from BD Bioscience. Anti-mouse-CD3-APC, Foxp3-PE, IL17-PE, anti-human-CD3-APC, CD4-APC, CD25-APC and Foxp3-PE antibodies were purchased from eBioscience. Anti-mouse IgG, Fas and Fas-ligand antibodies were purchased from Santa Cruz Biosciences. MCP-1 antibodies were purchased from Cell Signaling. Anti-rat-IgG-Rhodamine antibody was purchased from Southern Biotech. Anti-rat IgG-AlexaFluoro 488 antibody was purchased from Invitrogen. Anti-p-actin antibody was purchased from Sigma.

Isolation of Mouse Bone Marrow Mesenchymal Stem Cells (BMMSCs).

The single suspension of bone marrow-derived all nucleated cells (ANCs) from femurs and tibias were seeded at a density of 15×10⁶ in 100 mm culture dishes (Corning) under 37° C. at 5% CO2 condition. Non-adherent cells were removed after 48 hours and attached cells were maintained for 16 days in Alpha Minimum Essential Medium (a-MEM, Invitrogen) supplemented with 20% fetal bovine serum (FBS, Equitech-Bio, Inc.), 2 mM L-glutamine, 55 uM 2-mercaptoethanol, 100 U/ml penicillin, and 100 ug/ml streptomycin (Invitrogen). Colonies forming attached cells were passed once for further experimental use. Flow cytometric analysis showed that 0.95% of BMMSCs was positive for CD34⁺CD117⁺ antibody staining.

Isolation of Mouse B Cells, NK Cells, Immature Dendritic Cells (iDCs)/Macrophages.

After removing red blood cells using ACK lycing buffer, mouse splenocytes were incubated with anti-mouse CD19-PE, CD49b-FITC and CD11c-FITC antibodies for 30 min, followed by a magnetic separation using anti-PE or anti-FITC micro beads (Milteny biotech) according to manufacturer's instructions.

T Cell Culture.

Complete medium containing Dulbecco's Modified Eagle's Medium (DMEM, Lonza) with 10% heat-inactivated FBS, 50 ̂M 2-mercaptoethanol, 10 mM HEPES, 1 mM sodium pyruvate (Sigma), 1% non-essential amino acid (Cambrex), 2 mM L-glutamine, 100 U/ml penicillin and 100 mg/ml streptomycin.

Immunofluorescent Microscopy.

The macrophages or BMMSCs were cultured on 4-well chamber slides (Nunc) (2×10³/well) and then fixed with 4% paraformaldehyde. The chamber slides were incubated with primary antibodies including anti-CD11b antibody (1:400, BD), anti-CD90.2 (1:400, BD) and anti-FasL (1:200, SantaCruz) at 4° C. for overnight followed by treatment with Rhodamine-conjugated secondary antibody (1:400, Southern biotech) or AlexaFluoro 488-conjugated secondary antibody (1:200, Invitrogen) for 30 min at room temperature. Finally, slides were mounted with Vectashield mounting medium (Vector Laboratories).

Western Blotting Analysis.

Total protein was extracted using M-PER mammalian protein extraction reagent (Thermo). Nuclear protein was obtained using NE-PER nuclear and cytoplasmic extraction reagent (Thermo). Protein was applied and separated on 4-12% NuPAGE gel (Invitrogen) and transferred to Immobilon™-P membranes (Millipore). The membranes were blocked with 5% non-fat dry milk and 0.1% Tween 20 for 1 hour, followed by incubation with the primary antibodies (1:100-1000 dilution) at 40 C overnight. Horseradish peroxidase-conjugated IgG (Santa Cruz Biosciences; 1:10,000) was used to treat the membranes for 1 hour and subsequently enhanced with a SuperSignal® West Pico Chemiluminescent Substrate (Thermo). The bands were detected on BIOMAX MR films (Kodak). Each membrane was also stripped using a stripping buffer (Thermo) and re-probed with anti p-actin antibody to quantify the amount of loaded protein.

Real-Time Polymerase Chain Reaction (RT-PCR).

Total RNA was isolated from the cultures using SV total RNA isolation kit (Promega) and digested with DNase I, following the manufacturer's protocols. The cDNA was synthesized from 100 ng of total RNA using Superscript III (Invitrogen). PCR was performed using gene-specific primers and Cybergreen supermix (BioRad). RT-PCR was repeated in 3 independent samples. The gene-specific primer pairs are as follows: Human FasL (GeneBank accession number; NM_{—}000639.1, sense; 5′-CTCTTGAGCAGTCAGCAACAGG-3′, antisense; 5′-ATGGCAGCTGGTGAGTCAGG-3′), human Fas (GeneBank accession number; NM_{—}000043.4, antisense; 5′-CAACAACCATGCTGGGCATC-3′, sense; 5′-TGATGTCAGTCACTTGGGCATTAAC-3′), and human GAPDH (GeneBank accession number; NM_{—}002046.3, antisense; 5′-GCACCGTCAAGGCTGAGAAC-3′, sense; TGGTGAAGACGCCAGTGGA).

Enzyme-Linked Immunosorbent Assay (ELISA).

Peripheral blood samples were collected from mice using micro-hematocrit tubes with heparin (VWR) and centrifuged at 1000 g for 10 min to get serum samples. TGFp (eBioscience), mouse ANA, anti-dsDNA IgG and anti-dsDNA IgM (Alpha Diagnosis), human ANA (EUROIMMUN), mouse MCP-1, human MCP-1 (eBioscience) and creatinine (R&D Systems) levels were measured using a commercially available kit according to manufacturer's instructions. The results were averaged in each group. The intra-group differences were calculated between the mean values.

Depletion of Phagocytes.

To inhibit phagocytes, clodronate-liposome (200 nl/mouse; Encapsula Nano-Science, LLC) was injected into mice i.p. PBS-liposome was used as control.

Depletion of Tregs.

To inhibit Tregs differentiation in DSS-induced experimental colitis mice, anti-CD25 antibody (250̂g/mouse, biolegend) was administrated intraperitoneally after 3 days of DDS induction.

Cytokine Array Analysis.

Culture supernatants from BMMSC or lprBMMSC were analyzed using Mouse Cytokine Array Panel A Array Kit (R&D Systems) according to manufacturer's instructions. The results were scanned and analyzed using Image J software to calculate blot intensity. Cytokine array was repeated in 2 independent samples.

Immunohistochemistry Staining and TUNEL Staining.

For detection of CD3, femurs at 24 hours after BMMSC injection were harvested and used for paraffin embedded sections. For co-cultured sample, culture supernatant was removed and fixed by 1% paraformaldehyde at 4° C. overnight. The samples were blocked with serum matched to secondary antibodies, incubated with the CD3-specific antibodies (eBioscience, 1:400) 30 min at room temperature, and stained using VECTASTAIN Elite ABC Kit (UNIVERSAL) and ImmPACT VIP Peroxidase Substrate Kit (VECTOR), according to the manufacturers' instructions. For TUNEL staining, an apoptosis detection kit (Millipore) was used in accordance with the manufacturer's instructions, followed by TRAP staining and counterstaining with H&E. Three independent experiments were performed.

### Example I

### Fas Ligand (FasL) in BMMSCs Induces T Cell Apoptosis

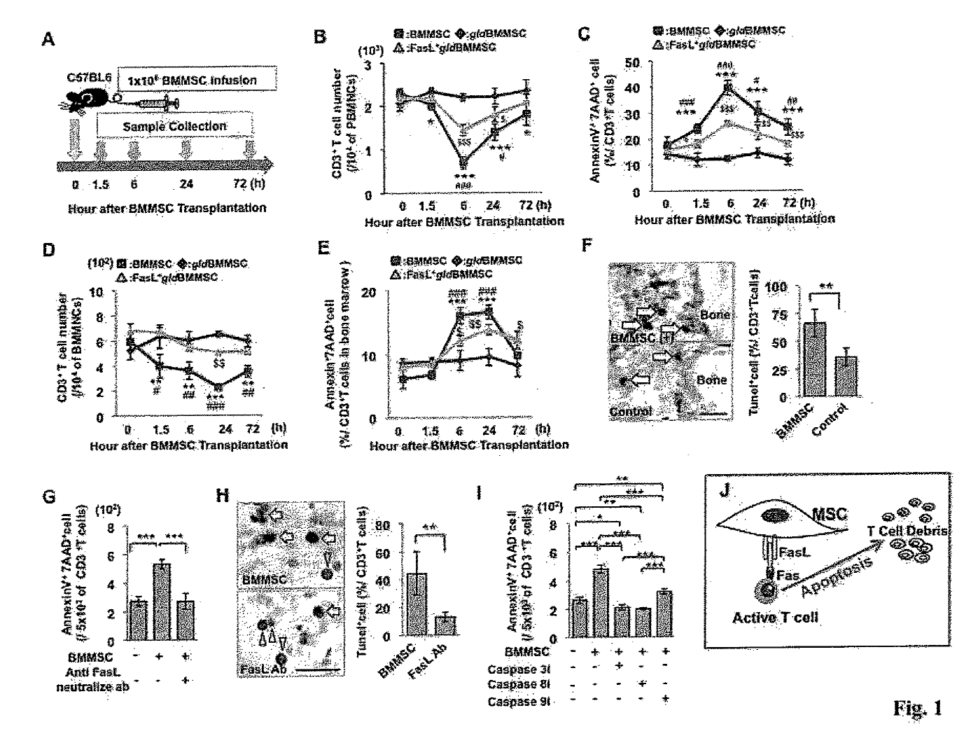

BMMSCs from C57BL6 mice and FasL-mutated B6Smn.C3-Fasl^{gld}/J mice (gldBMMSC), along with FasL transfected gldBMMSCs (FasL⁺gldBMMSC) were injected into normal C57BL6 mice (FIG. 1A). Similar to normal BMMSCs, FasL null gldBMMSCs express mesenchymal stem cell markers and possess multipotent differentiation capacity (data not shown). Peripheral blood and bone marrow samples were collected at 0, 1.5, 6, 24, and 72 hours after BMMSC transplantation for subsequent analysis (FIG. 1A). Allogenic BMMSC infusion reduced the number of CD3⁺ T cells and increased the number of apoptotic CD3⁺ T cells in peripheral blood and bone marrow, starting at 1.5 hours, reaching the peak at 6 hours and lasting until 72 hours post-transplantation (FIGS. 1B-1E). In order to compare syngenic and allogenic BMMSCs, we found that BMMSCs derived from a littermate are same as allogenic BMMSCs in inducing T cell apoptosis (FIGS. S2A-2G). Meanwhile, infusion of FasL^{-/-} gldBMMSCs failed to reduce the number of CD3⁺ T cells or elevate the number of apoptotic CD3⁺ T cells in peripheral blood and bone marrow (FIGS. 1B-1E). However, overexpression of FasL in gldBMMSCs by lentiviral transfection (FIG. 8N) rescued the capacity of BMMSCs to both reduce the number of CD3⁺ T cells and elevate the number of apoptotic CD3⁺ T cells in peripheral blood, bone marrow, spleen, and lymph node (FIGS. 1B-1E; S1P-1S). BMMSC infusion also resulted in reducing the number of both CD4⁺ and CD8⁺ T cells with correspondingly increased number of apoptotic CD4⁺ and CD8⁺ T cells in peripheral blood (FIGS. S1E and 1F). Interestingly, BMMSC transplantation induced CD4⁺ T cell apoptosis and Treg upregulation in OT1 TCR TG mice. However, the percentage of CD8⁺ T cells, which react with OVA-MHC class I antigen, was unchanged after BMMSC transplantation, indicating that transplanted BMMSCs need to be recognized as antigen to initiate CD8⁺ T cell apoptosis induction (FIGS. S1T-1AA). TUNEL staining confirmed that BMMSC infusion elevated the number of apoptotic T cells in bone marrow (FIG. 1F). We next verified that BMMSC-induced T cell death was caused by apoptosis based on the in vitro blockage of BMMSC-induced CD3⁺ T cell apoptosis by neutralizing FasL antibody and caspase 3, 8, and 9 inhibitors (FIGS. 1G-1I). FasL neutralizing antibody injection could partially block BMMSC-induced CD3⁺ T cell apoptosis, upregulation of Tregs, and downregulation of Th17 cells in peripheral blood and bone marrow (FIG. 8G-M). These data indicate that BMMSCs are capable of inducing T cell apoptosis through the FasL/Fas signaling pathway (FIG. 1J). In addition, BMMSC transplantation was capable of inducing transient CD19⁺ B cells and CD49b⁺ NK cells, but not CD11c⁺F4/80⁺ macrophage/immature dendritic cell apoptosis in C57BL6 mice (data not shown). Although BMMSCs failed to induce naïve T cell apoptosis in the co-culture system (data not shown), they were able to induce activated T cell apoptosis in vitro (FIGS. 1G and 1I).

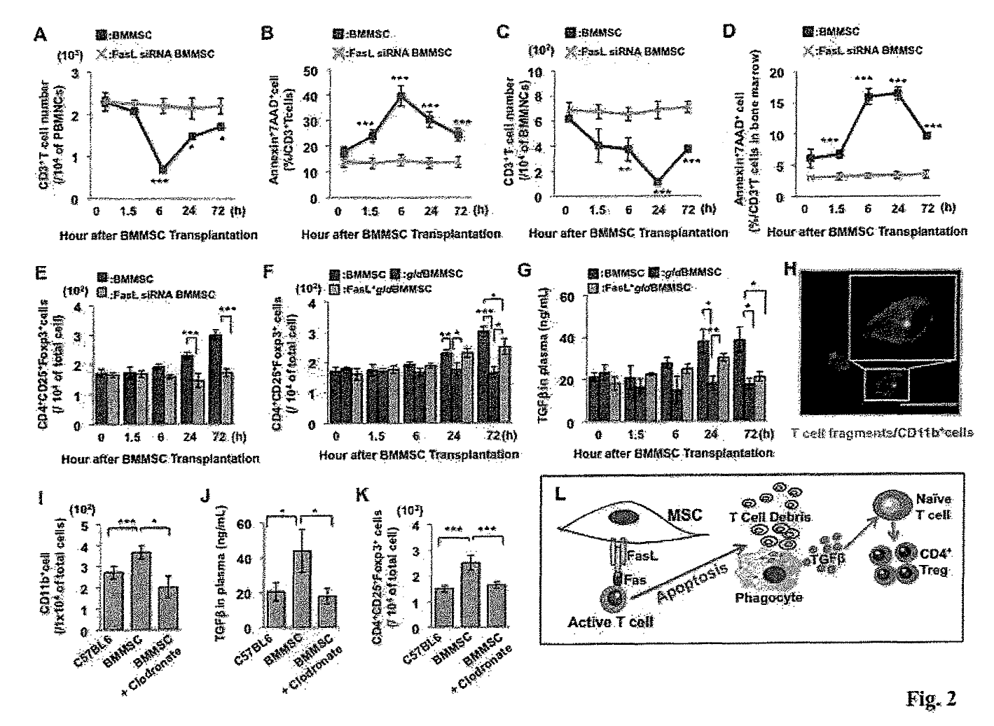

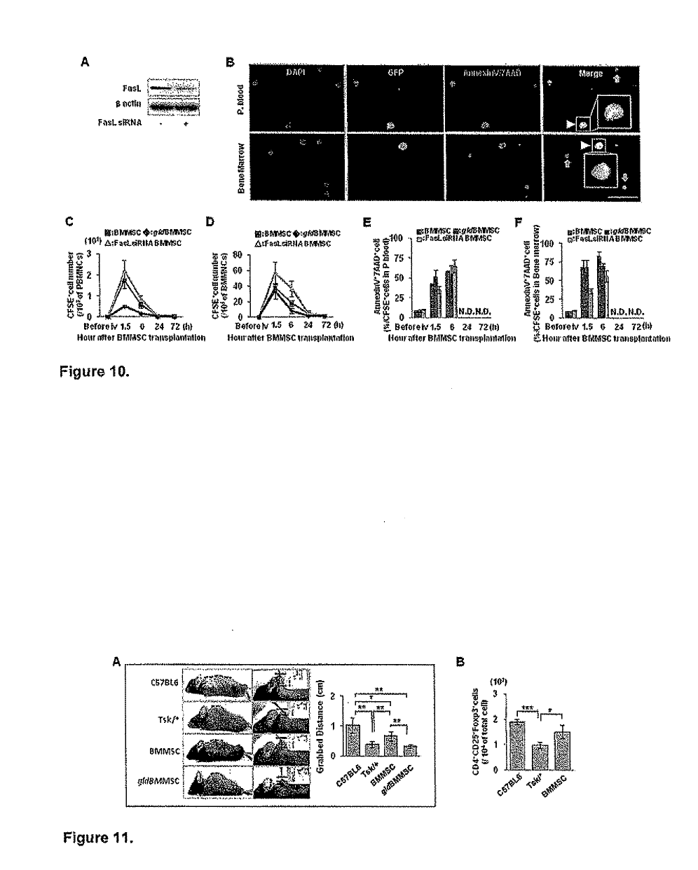

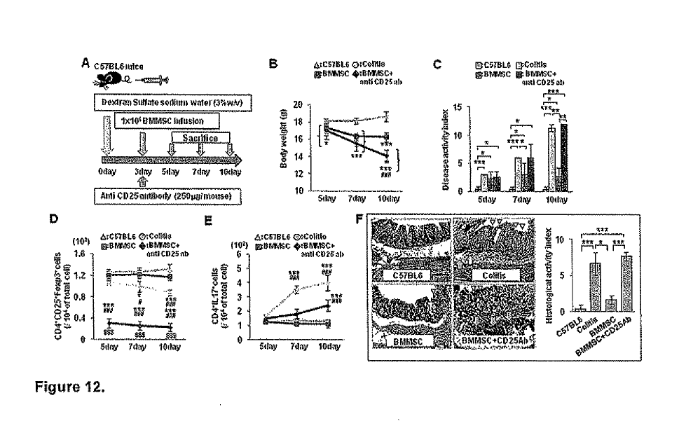

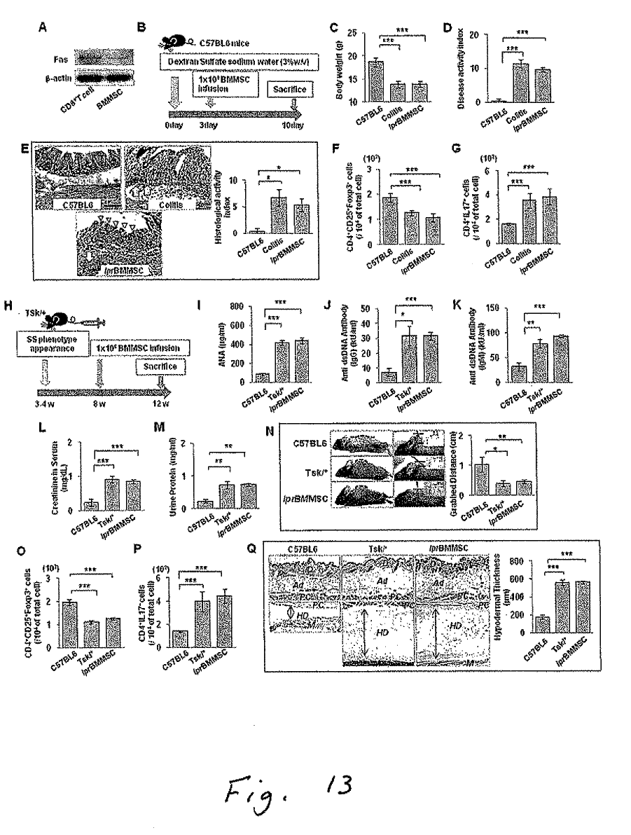

In order to confirm the role of FasL in BMMSC-mediated T cell apoptosis in vivo, we used siRNA to knockdown FasL expression in BMMSCs (FIG. 10A) and infused FasL knockdown BMMSCs to C57BL6 mice. Infusion of FasL knockdown BMMSCs (FasL siRNA BMMSCs) failed to reduce the number of CD3⁺ T cells or induce CD3⁺ T cell apoptosis in peripheral blood and bone marrow (FIGS. 2A-2D). Moreover, infusion of FasL knockdown BMMSCs failed to elevate CD4⁺CD25⁺Foxp3⁺ regulatory T cell (Treg) levels in peripheral blood (FIG. 2E). This study confirms that FasL is required for BMMSC-induced T cell apoptosis and Treg upregulation. Interestingly, six hours following initial BMMSC transplantation, we conducted a second transplantation of BMMSCs to C57BL6 mice and found that double BMMSC transplantation failed to further reduce the number of CD3⁺ T cells or upregulate Tregs compared to the single injection group (data not shown).

Since apoptotic T cells trigger TGF-β production by macrophages and up-regulates Tregs, which lead to immune tolerance in vivo (Perruche et al., 2008), we examined whether BMMSC-induced T cell apoptosis could also promote the upregulation of Tregs. We found that systemic infusion of mouse and human BMMSCs did, in fact, elevate Treg levels in peripheral blood at 24 and 72 hours post-transplantation (FIGS. 2F and S2H-2M), along with elevated TGF-β level and reduced T helper 17 (Th17) cell level in peripheral blood (FIGS. 2G and S10). Co-transplantation of BMMSCs and pan T cells resulted in significant T cell apoptosis at 1.5 and 6 hours post-transplantation. However, co-transplantation of BMMSCs with Tregs failed to significantly affect the level of Tregs, suggesting that BMMSC transplantation may not affect Treg survival (data not shown). In addition, we found that Tregs derived from BMMSC-transplanted and control mice showed the same rate of apoptosis under the apoptotic induction (data not shown). FasL^{-/-} gldBMMSC infusion failed to upregulate the levels of either Tregs or TGF-β (FIGS. 2F and 2G), suggesting that FasL-mediated T cell apoptosis plays a critical role in Treg upregulation. Indeed, overexpression of FasL in FasL^{-/-} gldBMMSCs rescued BMMSC-induced Treg upregulation and TGF-β production at 24 hours post-transplantation (FIGS. 2F and 2G).

To examine the mechanism by which BMMSC infusion resulted in TGF-β up-regulation in peripheral blood, we used fluorescence analysis to confirm that macrophages engulfed apoptotic T cells in vivo (Perruche et al., 2008; FIG. 2H). Then we measured the number of CD11b⁺ macrophages in spleen cells and found that the number was significantly increased in the BMMSC infusion group (FIG. 2I). In contrast, treatment with macrophage inhibitor clodronate liposomes significantly reduced the number of CD11b⁺ macrophages in spleen cells (FIG. 2I) and blocked BMMSC infusion-induced upregulation of TGF-β and Tregs (FIGS. 2J and 2K). However, injection of TGFβ failed to induce T cell apoptosis or upregulate Tregs in C57BL6 mice (data not shown), suggesting that elevated TGFβ level is not the only factor promoting Tregs in vivo. These data suggest that T cell apoptosis, as induced by BMMSC infusion, activates macrophages producing TGF-β, resulting in Treg upregulation (FIG. 2L).

We next asked whether apoptosis of infused BMMSCs also affects Treg upregulation. Carboxyfluorescein diacetate N-succinimidyl ester (CFSE)-labeled BMMSCs, gldBMMSCs and FasL knockdown BMMSCs were infused into C57BL6 mice. At 1.5 hours post-infusion, all CFSE cells were detected and reached a peak in peripheral blood and bone marrow, after which the cell number was gradually decreased, becoming undetectable at 24 hours post-infusion (FIGS. S3C and 3D). In contrast, CFSE⁺ apoptotic cells reached a peak at 6 hours post-infusion and became undetectable at 24 hours post-infusion in peripheral blood and bone marrow (FIGS. S3E and 3F). The apoptosis of transplanted BMMSCs was also observed by immunofluoresent analysis (FIG. 10B). Although apoptosis of the infused FasL-deficient BMMSCs was observed, there was no upregulation of TGF-β or Tregs in peripheral blood (FIGS. 2E, 2F, and 2O). These data suggest that T cell, not BMMSC, apoptosis is required for Treg upregulation (FIG. 2L).

### Example II

### FasL is Required for BMMSC-Based Immune Therapies in Both Tight-Skin (Tsk/⁺) Systemic Sclerosis and Inductive Experimental Colitis Mice

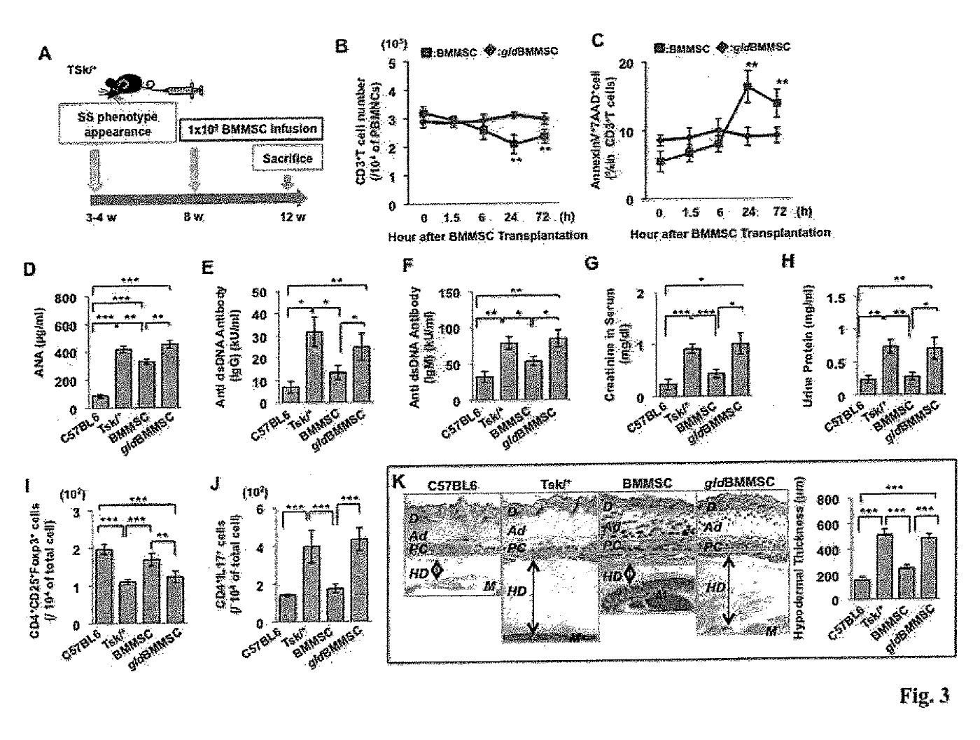

To further study the therapeutic mechanism of BMMSC transplantation, two mouse models, genetic tight-skin (Tsk/⁺) systemic sclerosis and inductive experimental colitis, were used to evaluate the therapeutic effect of BMMSC transplantation. Allogenic normal BMMSCs or gldBMMSCs (1×10⁶) were systemically transplanted into Tsk/⁺ systemic sclerosis mice (Green et al., 1976) at 8 weeks of age, and samples were harvested at 12 weeks of age for further evaluation (FIG. 3A). The BMMSC-transplanted group showed significant reduction in the number of CD3⁺ T cells and corresponding elevation in the number of apoptotic CD3⁺ T cells in peripheral blood from 6 to 72 hours post-transplantation (FIGS. 3B and 3C). On the other hand, FasL^{-/-} gldBMMSC transplantation failed to induce CD3⁺ T cell apoptosis (FIGS. 3B and 3C).

Tsk/⁺ mice showed an increase in the levels of anti nuclear antibody (ANA), anti-double strand DNA (dsDNA) IgG and IgM antibodies, and creatinine in serum, along with an increase in the level of urine proteins, at four weeks post-BMMSC transplantation (FIGS. 3D-3H). Normal BMMSC, but not FasL^{-/-} gldBMMSC, transplantation significantly reduced the levels of ANA, dsDNA IgG and IgM, as well as serum creatinine and urine protein levels (FIGS. 3D-3H). Moreover, BMMSC transplantation rescued decreased level of Tregs and increased level of Th17 cells in Tsk/⁺ mice (FIGS. 3I, 3J, and S4B). As expected, gldBMMSC transplantation failed to regulate the levels of Tregs and Th17 cells in Tsk/⁺ mice (FIGS. 3I and 3J). Histological analysis also showed that skin hypodermal (HD) thickness was significantly increased in Tsk/⁺ mice (FIG. 3K). After BMMSC transplantation, HD thickness was reduced to a level equal to that of the control group (C57BL6), whereas gldBMMSC failed to reduce HD thickness (FIG. 3K). Additionally, the tightness of skin, as measured by grabbed distance, was significantly improved in the BMMSC, but not the gldBMMSC, transplantation group (FIG. 11A).

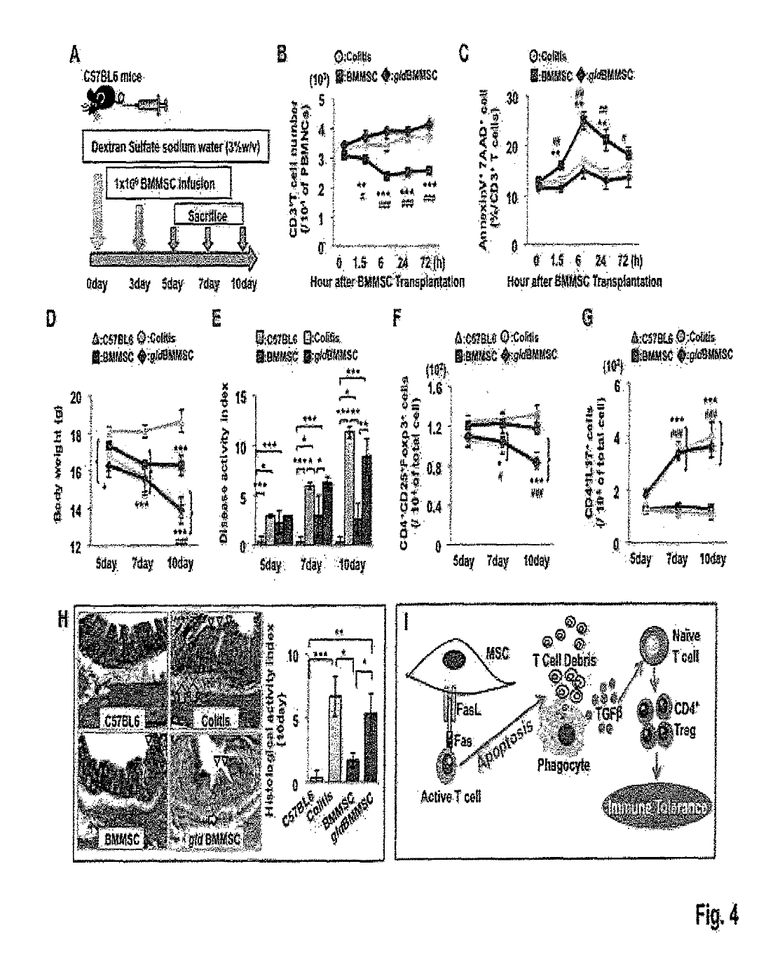

The induced experimental colitis model was generated as previously described (Alex et al., 2009; Zhang et al., 2010). Allogenic normal BMMSCs or FasL^{-/-} gldBMMSCs (1×10⁶) were systemically transplanted into experimental colitis mice at day 3 post 3% dextran sulfate sodium (DSS) induction (Zhang et al., 2010; FIG. 4A). Normal BMMSC transplantation reduced the number of CD3⁺ T cells and elevated the number of annexinV⁺7AAD⁺ double positive apoptotic CD3⁺ T cells in peripheral blood starting at 1.5 hours and lasting to 72 hours after transplantation (FIGS. 4B and 4C). However, the gldBMMSC transplantation group showed no difference from the colitis group in terms of numbers of CD3⁺ T cells and apoptotic CD3⁺ T cells (FIGS. 4B and 4C). The body weight of mice with induced colitis was significantly reduced compared to control C57BL6 mice from day 5 to 10 post-DSS induction (FIG. 4D). After normal BMMSC, but not gldBMMSC transplantation, the body weight was partially restored at day 10 post-DSS induction. The disease activity index (DAI), including body weight loss, diarrhea, and bleeding, was significantly elevated in the induced colitis mice compared to control mice. After BMMSC transplantation, the DAI score was decreased, while gldBMMSCs failed to reduce the DAI score (FIG. 4E). Both decreased Tregs and elevated Th17 cells were observed in the induced colitis mice from day 7 to 10 post-DSS induction (FIGS. 4F and 4O). BMMSC, but not gldBMMSC, transplantation significantly upregulated Tregs and downregulated Th17 cells (FIGS. 4F and 4G). Furthermore, colon tissue from each group was analyzed (FIG. 4H). Both the absence of epithelial layer and infiltration of inflammatory cells were observed in the induced colitis and gldBMMSC transplantation groups. BMMSC transplantation recovered epithelial structure and eliminated inflammatory cells in colitis mice. Histological activity index (Alex et al., 2009) confirmed that BMMSC transplantation reduced the DAI, while gldBMMSCs failed to improve the DAI (FIG. 4H). The data therefore suggest that BMMSC-induced T cell apoptosis with Treg upregulation might offer a potential treatment for induced colitis (FIG. 4I). Moreover, upregulation of Tregs was required in ameliorating disease phenotype in DSS-induced colitis model (FIGS. S5A-5F).

### Example III

### Fas is Required for BMMSC-Mediated Therapy by Recruitment of T Cells

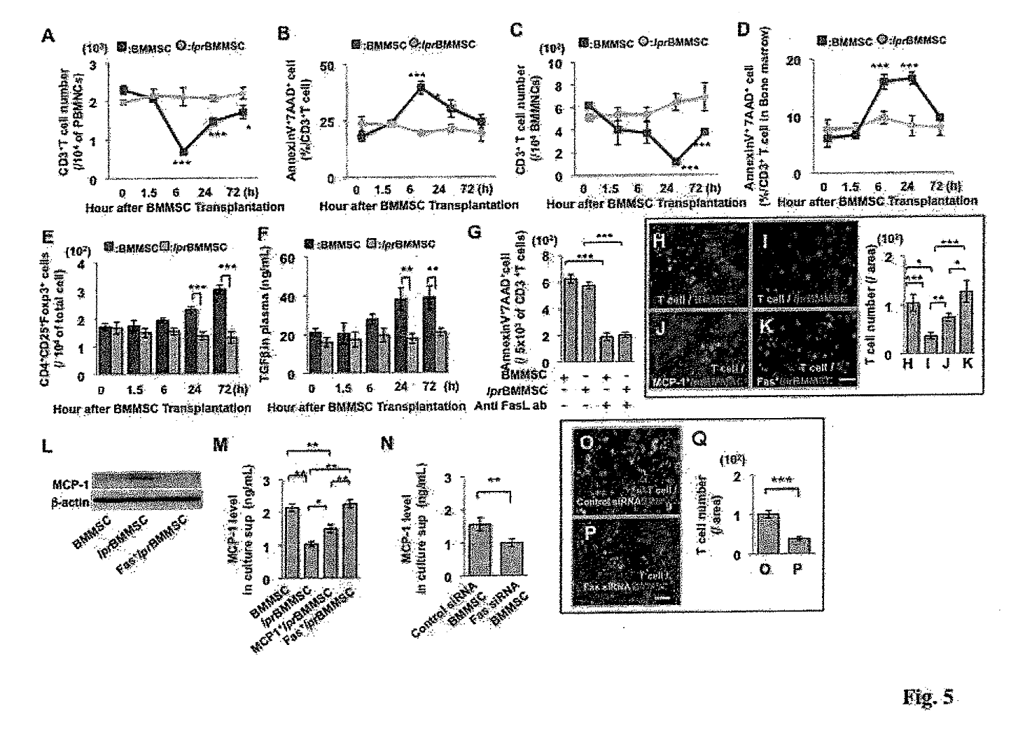

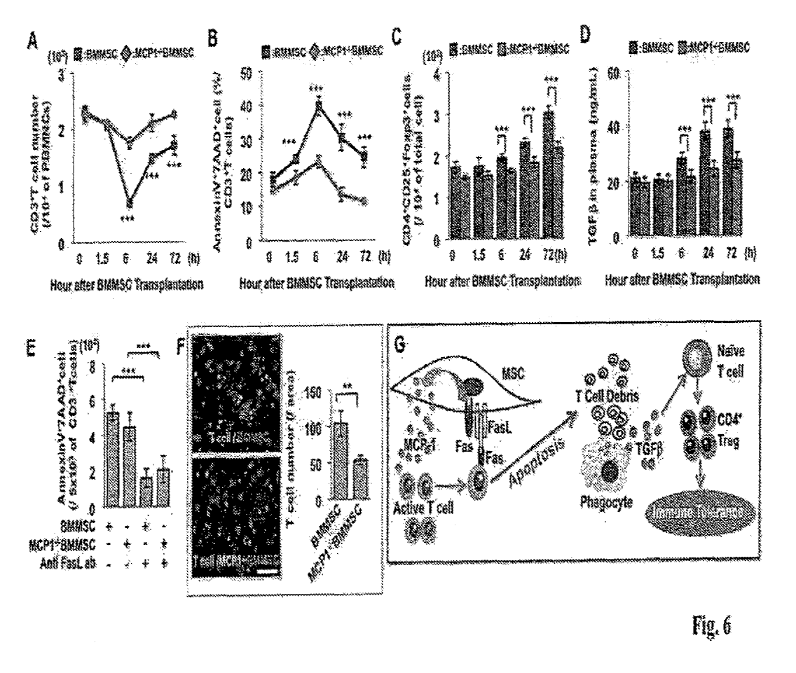

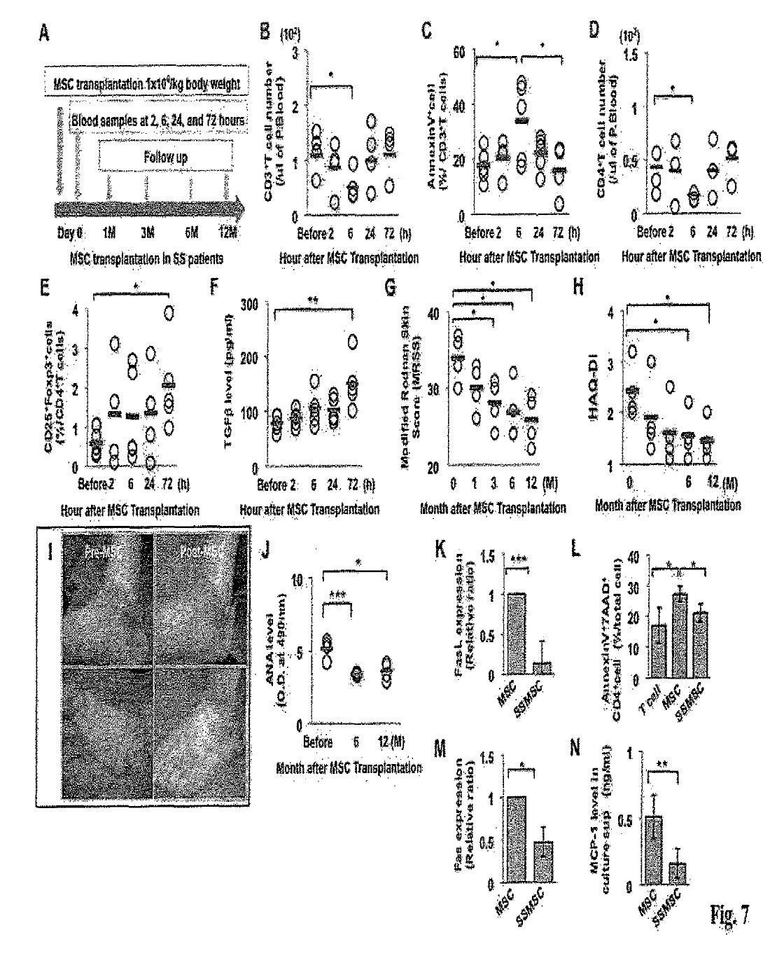

In addition to the production of FasL, the isolated BMMSCs used herein also express Fas (FIG. 13A). To examine whether Fas plays a role in BMMSC-based immunotherapies, we infused Fas^{-/-}BMMSCs, derived from C3MRL-Fas^{lpr}/J mice (lprBMMSCs), to C57BL6 mice and found that Fas^{-/-} lprBMMSCs failed to reduce number of CD3⁺ T cells or elevate the number of apoptotic CD3⁺ T cells in peripheral blood and bone marrow (FIGS. 5A-5D). As widely used autoimmune disease models, FasL null gld and Fas null lpr mice showed a significantly increased number of CD62L⁻CD44⁺ activated T cells and elevated ratio of Th1/Th2 and Th17/Treg (data not shown). In addition, both gld and lpr T cells showed reduced response to CD3 and CD28 antibody stimulation when compared to the control T cells (data not shown). It appeared that gld and lpr BMMSCs showed similar colony forming capacity, multipotent differentiation, and surface molecular expression (data not shown). In addition, we revealed that lprBMMSC transplantation failed to upregulate the levels of Tregs and TGF-β and downregulate Th17 cell level in peripheral blood (FIGS. 5E, 5F, and S6X). Moreover, Fas knockdown BMMSCs using siRNA showed the same effect as observed in Fas null lprBMMSC (FIG. 13Y-6EE). Although transplanted Fas null lprBMMSCs disappeared within 24 hours in peripheral blood, the number of AnnexinV/7AAD double positive BMMSCs was not significantly increased (data not shown), implying that another pathway may help to clear transplanted lprBMMSCs in recipient mice. When transplanted into DSS-induced colitis mice, lprBMMSCs failed to provide therapeutic effects on body weight, disease activity index, histological activity index, and lprBMMSCs were also unable to rebalance the levels of Tregs and Th17 cells (FIGS. S6B-6G). In addition, lprBMMSC transplantation failed to treat Tsk/⁺ SS mice, showing no rescue of the levels of ANA, anti-dsDNA antibodies IgG and IgM antibodies, creatinine, urine protein, Grabbed distance, Tregs, or Th17 cells (FIGS. S6H-6Q). Taken together, these data suggest that Fas^{-/-}lprBMMSCs, like FasL^{-/-} gldBMMSCs, were unable to ameliorate immune disorders in SS and colitis mouse models.

Next, we investigated the underlying mechanisms by which lprBMMSC transplantation failed to treat the diseases. We showed that lprBMMSCs expressed a normal level of FasL by Western blot analysis (FIG. 13R) and induced CD3⁺ T cell apoptosis in a co-culture system (FIG. 5G). This was blocked by anti-FasL neutralizing antibody (FIG. 5G), suggesting that the failure to induce in vivo T cell apoptosis by lprBMMSCs does not result from the lack of expression of functional FasL. We therefore hypothesized that Fas expression affects the BMMSC immunomodulatory property via a non-FasL-related mechanism, such as regulating the recruitment of T cells. To test this, we used an in vitro transwell co-culture system to show that activated T cells migrate to BMMSCs to initiate cell-cell contact (FIG. 5H). However, lprBMMSCs showed a significantly reduced capacity to recruit activated T cells in the co-culture system when compared to control BMMSCs (FIGS. 5H and 5I). We then used a cytokine array analysis to determine that lprBMMSCs express a low level of monocyte chemotactic protein 1 (MCP-1), a member of C-C motif chemokine family and a T cell chemoattractant cytokine (Carr et al. 1994; FIG. 13S). Interestingly, overexpression of MCP-1 in lprBMMSCs partially rescued their capacity to recruit T cells (FIGS. 5H-5J). Overexpression of Fas in lprBMMSCs showed that secretion level of multiple cytokine was restored (FIGS. S6S and S6U) and fully rescued their capacity to recruit T cells (FIGS. 5H, 5I, 5K). However, the expression level of MCP-1 protein in lprBMMSCs was higher than that in control BMMSCs, and overexpression of Fas reduced MCP-1 cytoplasm protein level in lprBMMSCs (FIG. 5L), indicating that Fas regulates MCP-1 secretion, but not expression. Next, we examined MCP-1 level in the culture supernatant, and we found that the MCP-1 level in lprBMMSCs was significantly lower than BMMSCs (FIG. 5M). Overexpression of MCP-1 and Fas in lprBMMSCs rescued MCP-1 levels in culture supernatant (FIG. 5M). We next confirmed that Fas regulated MCP-1 secretion using the siRNA knockdown approach (FIG. 13T). Down regulation of Fas expression in BMMSCs resulted in the reduction of MCP-1 secretion (FIG. 5N), with a corresponding reduction in the capacity to recruit activated T cells in the co-culture system (FIGS. 5O and 5P).

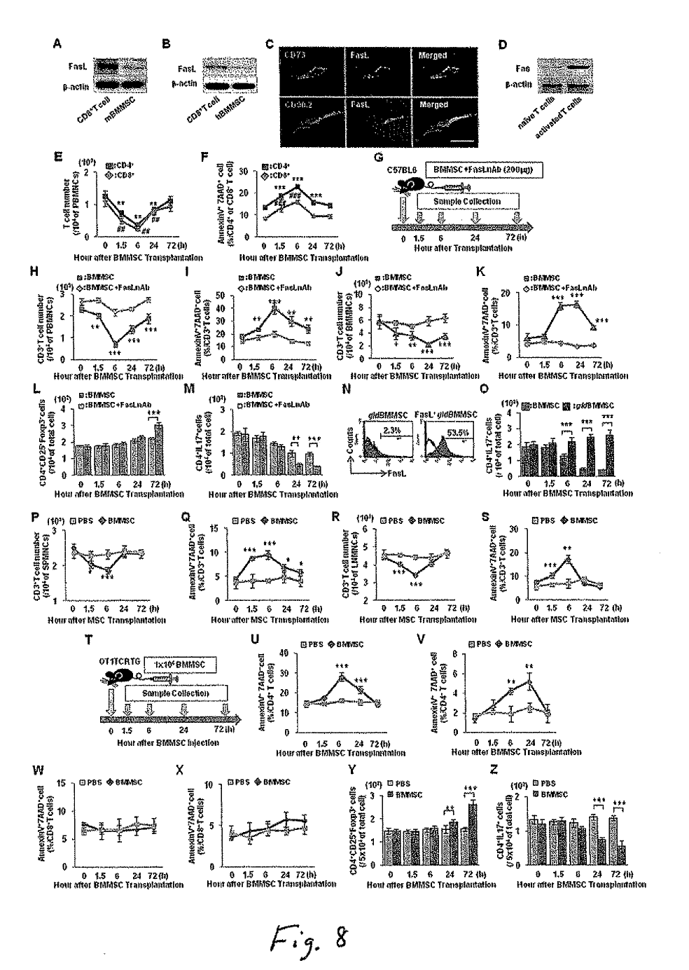

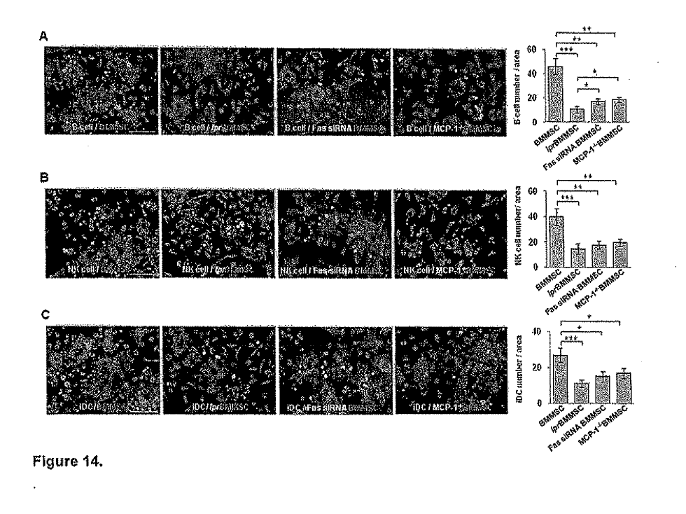

In order to confirm that MCP-1 contributes to BMMSC-based immunoregulation, we isolated BMMSCs from MCP-1 mutant B6.129S4-Ccl2^{tmlRol}/J mice and showed that MCP-1^{-/-} BMMSCs were defective in reducing the number of CD3⁺ T cells or elevating apoptotic CD3⁺ T cells in C57BL6 mice when compared to control BMMSCs (FIGS. 6A and 6B). Also, MCP-1^{-/-} BMMSCs failed to upregulate the levels of Tregs and TGF-β within 72 hours post-transplantation (FIGS. 6C and 6D). The deficiency of inducing T cell apoptosis and Treg up-regulation by MCP-1^{-/-} BMMSCs was not associated with FasL function (FIG. 6E). When MCP-1^{-/-} BMMSCs were co-cultured with activated T cells in a transwell culture system, the number of T cells migrating to BMMSCs was significantly reduced compared to control BMMSCs (FIG. 6F). Also, Fas and MCP-1 play an important role in attracting B cells, NK cells, and immature dendritic cells (iDCs) in an in vitro culture system (FIG. 14A-7C). These data indicate that MCP-1 secretion regulates BMMSC-induced T cell migration (FIG. 6G). Moreover, we showed that Fas also regulated the secretion of other cytokines, such as C-X-C motif chemokine 10 (CXCL-10) and tissue inhibitor of matrix metalloprotease-1 (TIMP-1) (FIGS. S6V and 6W).

### Example IV

### Allogenic MSC Transplantation (MSCT) Induced CD3⁺ T Cell Apoptosis and Treg Up-Regulation in Patients with Systemic Sclerosis (SS).

Based on the above results in experimental animal models, we conducted a pilot clinical investigation to assess whether T cell apoptosis and Treg upregulation occurred in SS patients treated with MSCT. Five patients (4 females and 1 male, Table S1), ranging in age from 44 to 61 years old (average 51.2±7.8 years old) and having SS for a duration of 48-480 months (average 163.2±182.1 months) were enrolled for allogenic MSCT and peripheral blood was collected at indicated time points (FIG. 7A). Allogenic MSC transplantation induced a significantly reduced number of CD3⁺ T cells and upregulated number of AnnexinV-positive apoptotic CD3⁺ T cells at 6 hours post-MSCT and then the CD3⁺ T cell number and apoptotic rate decreased to baseline level by 72 hours (FIGS. 7B and 7C). Reduced number of CD4⁺ T cells was also observed at 6 hours post-MSCT (FIG. 7D). Importantly, frequency of Tregs in peripheral blood was significantly upregulated at 72 hours post-MSCT (FIG. 7E), along with elevated level of TGFβ (FIG. 7F). Assessment of Modified Rodnan Skin Score (MRSS) and Health Assessment Questionnaire (HAQ-DI) indicated that MSCT provided optimal treatment for SS patients at follow-up period (FIGS. 7G and 7H). Furthermore, reduced level of ANA was observed in SS patients at 12 months follow up period (FIG. 7J). Interestingly, MSC derived from SS patient (SSMSC) showed deficiency in FasL and Fas expression when compared to MSC derived from healthy donors (MSC) (FIGS. 7K and 7M). SSMSCs showed a reduced capacity to induce T cell apoptosis (FIG. 7L) and to secrete MCP-1 (FIG. 7N), due to reduced expression levels of FasL and Fas. In addition, we found that MSCT significantly improved skin ulcers in a patient (FIG. 7I). These early clinical data demonstrate safety and efficacy of MSCT in SS patients and improvement of disease activities at post-allogenic MSCT. However, the long-term effects of MSCT on SS patients will require further investigation.

TABLE 1
SS Patient Information

| Patient | | | History of | | |
|---|---|---|---|---|---|
| | Age | | SS | | |
| No. | (years) | Gender | (months) | Clinical Symptom | Previous Treatments |
| 1 | 45 | M | 60 | RP, hardening Skin, | Predonison 7.5 mg/day, |
| | | | | ANA+, SCL70+ | Cyclosporin A 100 mg/ |
| | | | | | day |
| 2 | 58 | F | 480 | RP, hardening Skin, | Predonison 20 mg/day, |
| | | | | ANA+ | HCQ 400 mg/day |
| 3 | 61 | F | 72 | RP, hardening Skin, | Predonison 15 mg/day, |
| | | | | ANA+, SCL70+ | HCQ 400 mg/day |
| 4 | 44 | F | 156 | RP, hardening Skin, | Predonison 15 mg/day, |
| | | | | ANA+, SCL70+, anti | HCQ 400 mg/day |
| | | | | dsDNA+ | |
| 5 | 48 | F | 48 | RP, hardening Skin, | Predonison 5 mg/day, |
| | | | | ANA+ | Penicillamine 0.375 g/ |
| | | | | | day |

RP: Raynaud's phenomenon,
ANA: anti nuclear antibody,
SCL70: anti scleroderma antibody,
Anti dsDNA: anti double strand DNA antibody,
HCQ: Hydroxychloroquine.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Those skilled in the art will recognize, or be able to ascertain using no more than routine experimentation, many equivalents to the specific embodiments of the method and compositions described herein. Such equivalents are intended to be encompassed by the following claims.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

Herein is also made reference to “Mesenchymal-stem-cell-induced immunoregulation involves FAS-ligand−/Fas-mediated T cell apoptosis,” by Akiyama K., et al., Cell Stem Cell, May 4, 2012, vol. 10(5), pp. 544-555 (including supplementary information), the entire contents of which is hereby incorporated by reference.

### REFERENCES

The following references are incorporated herein in the entirety:
Aggarwal, S., and Pittenger, M F. (2005) Human mesenchymal stem cells modulate allogeneic immune cell responses. Blood 105, 1815-1822.
Akiyama, K., Chen, C., Wang, D., Xu, X., Qu, C., Yamaza, T., Cai, T., Chen, W., Sun, L., Shi, S., (2012) Mesenchymal-stem-cell-induced immunoregulation involves FAS-ligand−/Fas-mediated T cell apoptosis, Cell Stem Cell, 10(5), 544-555 (including supplementary information).
Alex, P., Zachos, N C., Nguyen, T., Gonzales, L., Chen, T E., Conklin, L S., Centola, M., Li, X. (2009) Distinct cytokine patterns identified from multiplex profiles of murine DSS and TNBS-induced colitis. Inflamm Bowel Dis. 15, 341-352.
Andersen, M H., Schrama, D., Thor Straten, P., Becker, J C. (2006). Cytotoxic T cells. J. Invest. Dermatol. 126, 32-41.
Augello, A., Tasso, R., Negrini, S M., Amateis, A., Indiveri, F., Cancedda, R., Pennesi, G. (2005) Bone marrow mesenchymal progenitor cells inhibit lymphocyte proliferation by activation of the programmed death 1 pathway. Eur. J. Immunol. 35, 1482-1490.
Augello, A., Tasso, R., Negrini, S. M., Cancedda, R., Pennesi, G. (2007) Cell therapy using allogeneic bone marrow mesenchymal stem cells prevents tissue damage in collagen-induced arthritis. Arthritis Rheum. 56, 1175-1186.
Batten, P., Sarathchandra, P., Antoniw, J W., Tay, S S., Lowdell, M W., Taylor, P M., and Yacoub, M H. (2006). Human mesenchymal stem cells induce T cell anergy and downregulate T cell allo-responses via the TH2 pathway: Relevance to tissue engineering human heart valves. Tissue Eng. 12, 2263-2273.
Bernardo, M E., Locatelli, F., Fibbe, W E. (2009) Mesenchymal stromal cells. Ann N Y Acad Sci. 1176, 101-117.
Carr, M W., Roth, S J., Luther, E., Rose, S S., Springer, T A. (1994) Monocyte chemoattractant protein 1 acts as a T-lymphocyte chemoattractant. Proc Natl Acad Sci USA. 91, 3652-3656.
Chatenoud, L., Thervet, E., Primo, J., Bach, J F. (1994) Anti-CD3 antibody induces long-term remission of overt autoimmunity in nonobese diabetic mice. Proc Natl Acad Sci USA. 91, 123-127.
Chatenoud, L., Primo, J., Bach, J F. (1997) CD3 antibody-induced dominant self tolerance in overtly diabetic NOD mice. J Immunol. 158, 2947-2954.
Chen, X., Armstrong, M. A., Li, G. (2006) Mesenchymal stem cells in immunoregulation. Immunol. Cell Biol. 84, 413-421.
Choi, H., Lee, R. H., Bazhanov, N., Oh, J. Y., Prockop, D. J. (2011) Anti-inflammatory protein TSG-6 secreted by activated MSCs attenuates zymosan-induced mouse peritonitis by decreasing TLR2/NF-{kappa}B signaling in resident macrophages. Blood. 118, 330-338.
Corcione, A., Benvenuto, F., Ferretti, E., Giunti, D., Cappiello, V., Cazzanti, F., Risso, M., Gualandi, F., Mancardi, G. L., Pistoia, V., Uccelli, A. (2006) Human mesenchymal stem cells modulate B-cell functions. Blood 107, 367-372.
González, M A., Gonzalez-Rey, E., Rico, L., Büscher, D., Delgado, M. (2009) Adipose-derived mesenchymal stem cells alleviate experimental colitis by inhibiting inflammatory and autoimmune responses. Gastroenterology 136, 978-989.
Green, M C., Sweet, H O., Bunker, L E. (1976) Tight-skin, a new mutation of the mouse causing excessive growth of connective tissue and skeleton. Am. J. Pathol. 82, 493-512.
Hohlbaum, A M., Moe, S. & Marshak-Rothstein, A. (2000) Opposing effects of transmembrane and soluble Fas ligand expression on inflammation and tumor cell survival. J. Exp. Med. 191, 1209-1219.
Kikuiri, T., Kim, I., Yamaza, T., Akiyama, K., Zhang, Q., Li, Y., Chen, C., Chen, W., Wang, S., Le, A D., Shi, S. (2010) Cell-based immunotherapy with mesenchymal stem cells cures bisphosphonate-related osteonecrosis of the jaw-like disease in mice. J Bone Miner Res. 25. 1668-1679.
Kleinclauss, F., Perruche, S., Masson, E., Carvalho-Bittencourt, M., Biichle, S., Remy-Martin, J P, Ferrand, C., Martin, M., Bittard, H., Chalopin, J M., Seilles, E., Tiberghien, P., Saas, P. (2006) Intravenous apoptotic spleen cell infusion induces a TGF-beta-dependent regulatory T-cell expansion. Cell Death Differ. 18, 41-52.
Le Blanc, K., Frassoni, F., Ball, L., Locatelli, F., Roelofs, H., Lewis, I., Lanino, E., Sundberg, B., Bernardo, M. E., Remberger, M., Dini, G., Egeler, R. M., Bacigalupo, A., Fibbe, W., Ringdén, O. (2004) Treatment of severe acute graft-versus-host disease with third party haploidentical mesenchymal stem cells. Lancet 363, 1439-1441.
Lee, R H., Seo, M J., Reger, R L., Spees, J L., Pulin, A A., Olson, S D., Prockop, D J. (2006) Multipotent stromal cells from human marrow home to and promote repair of pancreatic islets and renal glomeruli in diabetic NOD/scid mice. Proc. Natl. Acad. Sci. USA 103, 17438-17443.
Liang, J., Zhang, H., Hua, B., Wang, H., Wang, J., Han, Z., Sun, L. (2009) Allogeneic mesenchymal stem cells transplantation in treatment of multiple sclerosis. Mult. Scler. 15, 644-646.
Liang, J., Gu, F., Wang, H., Hua, B., Hou, Y., Shi, S., Lu, L., Sun, L. (2010) Mesenchymal stem cell transplantation for diffuse alveolar hemorrhage in SLE. Nat Rev Rheumatol. 6, 486-489.
Liang, J., Zhang, H., Wang, D., Feng, X., Wang, H., Hua, B., Liu, B., Sun, L. (2012) Allogeneic mesenchymal stem cell transplantation in seven patients with refractory inflammatory bowel disease. Gut. 61(3), 468-469.
Liang, J., Li, X., Zhang, H., Wang, D., Feng, X., Wang, H., Hua, B., Liu, B., Sun, L. (2012) Allogeneic mesenchymal stem cells transplantation in patients with refractory RA. Clin Rheumatol. 31, 157-161.
Mazar, J., Thomas, M., Bezrukov, L., Chanturia, A., Pekkurnaz, G., Yin, S., Kuznetsov, S., Robey, P G., and Zimmerberg, J (2009) Cytotoxicity Mediated by the Fas Ligand (FasL)-activated Apoptotic Pathway in Stem Cells. J. Biol. Chem. 284, 22022-22028.
Meisel, R., Zibert, A., Laryea, M., Go\ddot{}bel, U., Da\ddot{}ubener, W., and Dilloo, D. (2004). Human bone marrow stromal cells inhibit allogeneic T-cell responses by indoleamine 2,3-dioxygenase-mediated tryptophan degradation. Blood 103, 4619-4621.
Nauta, A J., Fibbe, W E. (2007) Immunomodulatory properties of mesenchymal stromal cells. Blood 110, 3499-3506.
Nasef, A., Mazurier, C., Bouchet, S. Francois, S., Chapel, A., Thierry, D., Gorin, N C., Fouillard, L. (2008) Leukemia inhibitory factor: Role in human mesenchymal stem cells mediated immunosuppression. Cell Immunol. 253, 16-22.
Németh, K., Leelahavanichkul, A., Yuen, P. S., Mayer, B., Parmelee, A., Doi, K., Robey, P. G., Leelahavanichkul, K., Koller, B. H., Brown, J. M., Hu, X., Jelinek, I., Star, R. A., Mezey, E. (2009) Bone marrow stromal cells attenuate sepsis via prostaglandin E (2)-dependent reprogramming of host macrophages to increase their interleukin-10 production. Nat. Med. 15, 42-49.
Parekkadan, B., Tilles, A W., Yarmush, M L. (2008) Bone marrow-derived mesenchymal stem cells ameliorate autoimmune enteropathy independent of regulatory T cells. Stem Cells 26, 1913-1919.
Park, M J., Park, H S., Cho, M L., Oh, H J., Cho, Y G., Min, S Y., Chung, B H., Lee, J W., Kim, H Y., Cho, S G. (2011) Transforming growth factor β-transduced mesenchymal stem cells ameliorate experimental autoimmune arthritis through reciprocal regulation of Treg/Th17 cells and osteoclastogenesis. Arthritis Rheum. 63, 1668-1680.
Perruche, S., Zhang, P., Liu, Y., Saas, P., Bluestone, J A., Chen, W. (2008) CD3-specific antibody-induced immune tolerance involves transforming growth factor-beta from phagocytes digesting apoptotic T cells. Nat Med. 5, 528-535.
Pluchino, S., Zanotti, L., Rossi, B., Brambilla, E., Ottoboni, L., Salani, G., Martinello, M., Cattalini, A., Bergami, A., Furlan, R., Comi, G., Constantin, G., Martino, G. (2005) Nature 436, 266-271.
Plumas, J., Chaperot, L., Richard, M J., Molens, J P., Bensa, J C., Favrot, M C. (2005) Mesenchymal stem cells induce apoptosis of activated T cells. Leukemia. 2005, 19. 1597-1604.
Polchert, D., Sobinsky, J., Douglas, G W., Kidd, M., Moadsiri, A., Reina, E., Genrich, K., Mehrotra, S., Setty, S., Smith, B., Bartholomew, A. (2008) IFN-γ activation of mesenchymal stem cells for treatment and prevention of graft versus host disease. Eur. J. Immunol. 38, 1745-1755.
Rafei, M., Campeau, P M., Aguilar-Mahecha, A., Buchanan, P W., Birman, E., Yuan, S., Young, Y K., Boivin, M N., Former, K., Basik, M., Galipeau, J. (2009) Mesenchymal stromal cells ameliorate experimental autoimmune encephalomyelitis by inhibiting CD4 Th17 T cells in a CC chmokine ligand 2-dependent manner. J. Immunol. 182, 5994-6002.
Ren, G., Zhang, L., Zhao, X., Xu, G., Zhang, Y., Roberts, A. I., Zhao, R. C., Shi, Y. (2008) Mesenchymal stem cell-mediated immunosuppression occurs via concerted action of chemokines and nitric oxide. Cell Stem Cell. 2, 141-150.
Ren, G., Zhao, X., Zhang, L., Zhang, J., L'Huillier, A., Ling, W., Roberts, A I., Le, A D., Shi, S., Shao, C., Shi, Y. (2010) Inflammatory cytokine-induced intercellular adhesion molecule-1 and vascular cell adhesion molecule-1 in mesenchymal stem cells are critical for immunosuppression. J Immunol. 184, 2321-2328.
Roddy, G. W., Oh, J. Y., Lee, R. H., Bartosh, T. J., Ylostalo, J., Coble, K., Rosa, R. H. Jr, Prockop, D. J. (2011) Action at a Distance: Systemically Administered Adult Stem/Progenitor Cells (MSCs) Reduce Inflammatory Damage to the Cornea Without Engraftment and Primarily by Secretion of TSG-6. Stem Cells. August 11. doi: 10.1002/stem.708. [Epub ahead of print]
Sato, K., Ozaki, K., Oh, I., Meguro, A., Hatanaka, K., Nagai, T., Muroi, K., Ozawa, K. (2007) Nitric oxide plays a critical role in suppression of T-cell proliferation by mesenchymal stem cells. Blood 109, 228-234.
Selmani, Z., Naji, A., Zidi, I., Favier, B., Gaiffe, E., Obert, L., Borg, C., Saas, P., Tiberghien, P., Rouas-Freiss, N., Carosella, E. D., Deschaseaux, F. (2008) Human leukocyte antigen-G5 secretion by human mesenchymal stem cells is required to suppress T lymphocyte and natural killer function and to induce CD4+CD25highFOXP3+ regulatory T cells. Stem Cells 26, 212-222.
Schurgers, E., Kelchtermans, H., Mitera, T., Geboes, L., and Matthys, P., (2010) Discrepancy between the in vitro and in vivo effects of murine mesenchymal stem cells on T-cell proliferation and collagen-induced arthritis. Arthritis Res Ther. 12, R31.
Spaggiari, G. M., Capobianco, A., Abdelrazik, H., Becchetti, F., Mingari, M. C., Moretta, L. (2008) Mesenchymal stem cells inhibit natural killer-cell proliferation, cytotoxicity, and cytokine production: role of indoleamine 2,3-dioxygenase and prostaglandin E2. Blood 111, 1327-1333.
Sun, L., Akiyama, K., Zhang, H., Yamaza, T., Hou, Y., Zhao, S., Xu, T., Le, A., Shi, S. (2009) Mesenchymal Stem Cell Transplantation Reverses Multi-Organ Dysfunction in Systemic Lupus Erythematosus Mice and Humans. Stem Cells 27, 1421-1432.
Uccelli, A., Moretta, L., Pistoia, V. (2008) Mesenchymal stem cells in health and disease. Nat Rev Immunol 8, 726-736.
Uccelli, A., Pistoia, V., Moretta, L. (2007) Mesenchymal stem cells: a new strategy for immunosuppression? Trends Immunol. 28, 219-226.
Wang, D., Zhang, H., Cao, M., Tang, Y., Liang, J., Feng, X., Wang, H., Hua, B., Liu, B., Sun, L. (2011) Efficacy of allogeneic mesenchymal stem cell transplantation in patients with drug-resistant polymyositis and dermatomyositis. Ann Rheum Dis. 70, 1285-1288.
Xu, L L., Warren, M K., Rose, W L., Gong, W., Wang, J M. (1996) Human recombinant monocyte chemotactic protein and other C-C chemokines bind and induce directional migration of dendritic cells in vitro. J. Leukoc Biol. 60, 365-371.
Zappia, E., Casazza, S., Pedemonte, E., Benvenuto, F., Bonanni, I., Gerdoni, E., Giunti, D., Ceravolo, A., Cazzanti, F., Frassoni, F., Mancardi, G., Uccelli, A. (2005) Mesenchymal stem cells ameliorate experimental autoimmune encephalomyelitis inducing T cell anergy. Blood 106, 1755-1761.
Zhang, Q., Shi, S., Liu, Y., Uyanne, J., Shi, Y., Shi, S., Le, A D. (2010) Mesenchymal stem cells derived from human gingiva are capable of immunomodulatory functions and ameliorate inflammation-related tissue destruction in experimental colitis. J Immunol. 184, 1656-1662.
Zhang, Y., Xu, G., Zhang, L., Roberts, Al., Shi, Y. (2008) Th17 cells undergo Fas-mediated activation-induced cell death independent of IFN-gamma. J Immunol. 181, 190-196.
Zhou, K., Zhang, H., Jin, O., Feng, X., Yao, G., Hou, Y., Sun, L. (2008) Transplantation of human bone marrow mesenchymal stem cell ameliorates the autoimmune pathogenesis in MRL/lpr mice. Cell Mol. Immunol. 5, 417-424.

1. A method of treating a patient comprising administering a composition comprising a therapeutically effective amount of an isolated and purified population of mesenchymal stem cells (MSCs) to the patient, wherein said MSCs: a) express Fas, b) express FasL and c) secrete MCP-1.

2. (canceled)

3. The method of claim 1, wherein said MSCs are bone marrow MSCs (BMMSCs).

4. The method of claim 3, wherein said BMMSCs are human BMMSCs (hBMMSCs).

5. The method of claim 1, wherein said MSCs are allogenic.

6. The method of claim 1, wherein from 1×10³ to 1×10⁷ of said MSCs per kg body weight of the patient are administered.

7. The method of claim 1, wherein from 1×10 to 1×10⁷ of said MSCs per kg body weight of the patient are administered.

8. The method of claim 1, wherein said MSCs are administered by infusion.

9. The method of claim 1, wherein said MSCs are administered by transplantation.

10-22. (canceled)

23. An isolated and purified population of MSCs, wherein said MSCs a) express Fas, b) express FasL and c) secrete MCP-1.

24. The isolated and purified population of MSCs of claim 23, wherein the MSCs are bone marrow mesenchymal stem cells (BMMSCs).

25. The isolated and purified population of MSCs of claim 24, wherein the BMMSCs are human BMMSCs.

26. The isolated and purified population of MSCs of claim 23, wherein said MSCs have been transfected with a vector comprising a gene for human FasL operably linked to a promoter, and wherein FasL is overexpressed from said vector.

27. The isolated and purified population of MSCs of claim 26, wherein said MSCs have been transfected with a vector comprising a gene for human Fas operably linked to a promoter, and wherein Fas is overexpressed from said vector.

28-43. (canceled)

44. A pharmaceutical composition comprising the isolated and purified population of MSCs of claim 23 dispersed in a pharmaceutically acceptable carrier.

45. The method of claim 1, wherein the patient is a patient with an inflammatory disease and/or an autoimmune disease.

46. The method of claim 1, wherein the patient is a patient with systemic sclerosis.

47. The method of claim 1, wherein the patient is a patient with colitis.

48. The method of claim 1, wherein the method produces immune tolerance to immunotherapies in the patient.

49. The method of claim 1, wherein said administration causes an upregulation in the level of regulatory T cells in the peripheral blood of the patient.
